(19) United States
(12) Patent Application Publication
(10) Pub. No.: US 2024/0428954 A1
(43) Pub. Date: Dec. 26, 2024

(54) SYSTEMS AND METHODS FOR DIAGNOSING SPINAL CONDITIONS AND DETERMINING TREATMENT OF THE SAME

(71) Applicant: Carlsmed, Inc., Carlsbad, CA (US)

(72) Inventor: Niall Patrick Casey, Carlsbad, CA (US)

(21) Appl. No.: 18/752,624

(22) Filed: Jun. 24, 2024

Related U.S. Application Data
(60) Provisional application No. 63/522,815, filed on Jun. 23, 2023.

(51) Int. Cl. G16H 50/50; G16H 10/60; G16H 20/40; G16H 30/40; G16H 50/70

(52) U.S. Cl. CPC G16H 50/50; G16H 10/60; G16H 20/40; G16H 30/40; G16H 50/70

(57) ABSTRACT

The present technology includes systems and methods for generating and implementing patient-specific diagnostic plans. The diagnostic plans include one or more specific diagnostic tests that should be performed on a particular patient based on the patient's demographics and/or health complaints. The diagnostic tests can be selected to determine specific diagnostic information that is expected to be useful in determining patient-specific surgical plans for the particular patient.

### CROSS-REFERENCE TO RELATED APPLICATION

The present application claims priority to U.S. Provisional Patent Application No. 63/522,815, filed Jun. 23, 2023, the disclosure of which is incorporated by reference herein in its entirety.

### TECHNICAL FIELD

The present disclosure is generally related to medical care, and more particularly to patient-specific medical implants, including systems and methods designing and manufacturing the same.

### BACKGROUND

Surgical procedures to implant orthopedic implants are used to correct numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper- or hypo-), and irregular spinal displacement (e.g., spondylolisthesis). Other spinal disorders that can be treated using an orthopedic implant include osteoarthritis, lumbar degenerative disc disease or cervical degenerative disc disease, lumbar spinal stenosis, and cervical spinal stenosis.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a network connection diagram illustrating a system for providing patient-specific medical care in accordance with embodiments of the present technology.

FIG. 2 illustrates a computing device suitable for use in connection with the system of FIG. 1 in accordance with embodiments of the present technology.

FIG. 3 is a flow diagram illustrating a method for determining and implementing a patient-specific diagnostic plan in accordance with embodiments of the present technology.

FIGS. 4A and 4B are tables listing representative diagnostic tests that can be prescribed as part of the patient-specific diagnostic plans generated in accordance with embodiments of the present technology.

FIG. 5 is a flow diagram illustrating a method for providing patient-specific medical care in accordance with embodiments of the present technology.

### DETAILED DESCRIPTION

The present technology includes systems and method for providing patient-specific medical care. In many of the embodiments described herein, providing patient-specific medical care includes generating and implementing a patient-specific diagnostic plan. The patient-specific diagnostic plans can prescribe one or more specific diagnostic tests that should be performed on a particular patient based on the patient's demographics and/or health. The one or more specific diagnostic tests can also be based at least in part on other factors, such as availability of diagnostic equipment, cost, convenience to patient, physician comfortability with various diagnostic procedures, or the like.

The diagnostic tests can be selected to determine specific diagnostic information that is expected to be useful in determining patient-specific surgical plans. The diagnostic information can include the results of the one or more diagnostic tests, and/or analysis of the results of the one or more diagnostic tests. Without being bound by theory, identifying patient-specific diagnostic plans in accordance with the present technology is expected to (1) preserve medical resources and lower costs by reducing the amount of unnecessary diagnostic tests performed on patients, and (2) improve patient outcomes by ensuring that the correct diagnostic tests are performed on the patient, and therefore appropriate diagnostic information is obtained, before determining a treatment plan for the patient.

In some embodiments, the systems and methods described herein include creating a virtual biomechanical model of a target anatomical region, such as the patient's spine, based on diagnostic information obtained via the patient-specific diagnostic plan. In some embodiments, one or more surgical procedures can be simulated on the virtual biomechanical model to generate simulated procedure data. The simulated procedure data can be compared with reference diagnostic data (e.g., prior patient data of similar patients) to determine one or more recommended surgical procedures for the patient. A surgical plan can then be generated based on the determined surgical procedure. In this way, the diagnostic information collected via execution of the patient-specific diagnostic plan can be used to generate patient-specific medical treatment plans.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

The headings are provided for convenience only and should not be used to interpret the scope of the present technology.

### A. Select Embodiments of Systems for Designing Patient-Specific Surgical Plans and Patient-Specific Implants

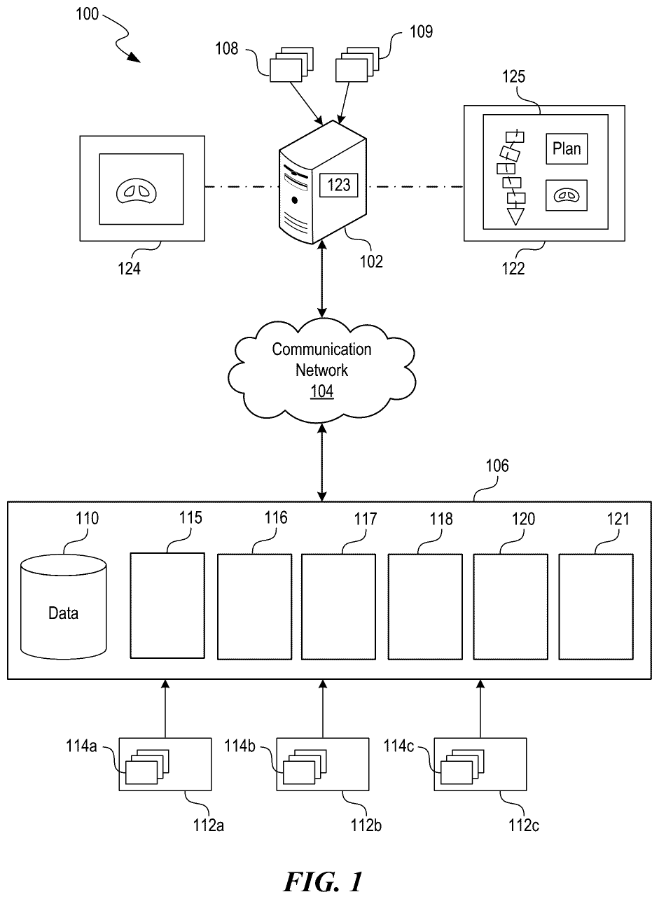

FIG. 1 is a network connection diagram illustrating a system 100 for providing patient-specific medical care, according to embodiments of the present technology. As described in further detail herein, the system 100 is configured to generate a medical treatment plan for a patient, including by developing and implementing a patient-specific diagnostic plan. In some embodiments, the system 100 is configured to generate a diagnostic plan and/or treatment plan for a patient suffering from an orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement axial displacement), osteoarthritis, lumbar degenerative disc disease, cervical degenerative disc disease, lumbar spinal stenosis, or cervical spinal stenosis, or a combination thereof. The diagnostic plan can include specific diagnostic tests to be performed on the patient to collet diagnostic information. The treatment plan can include surgical information, technology recommendations (e.g., device and/or instrument recommendations), and/or medical device designs based at least in part on the collected diagnostic information. For example, the medical treatment plan can include at least one surgical procedure (e.g., a surgical procedure or intervention) and/or at least one medical device (e.g., an implanted medical device (also referred to herein as an “implant” or “implanted device”) and/or implant delivery instrument). In some embodiments, the treatment plan is therefore also referred to as a “surgical plan,” a “patient-specific surgical plan,” a “patient-specific treatment plan,” or the like.

In some embodiments, the system 100 generates a medical treatment plan that is customized for a particular patient or group of patients, also referred to herein as a “patient-specific” or “personalized” treatment or surgical plan. The patient-specific surgical plan can include at least one patient-specific surgical procedure and/or at least one patient-specific medical device that are designed and/or optimized for the patient's particular characteristics (e.g., condition, anatomy, pathology, condition, medical history). For example, the patient-specific medical device can be designed and manufactured specifically for the particular patient, rather than being an off-the-shelf device. However, it shall be appreciated that a patient-specific surgical plan can also include aspects that are not customized for the particular patient. For example, a patient-specific or personalized surgical procedure can include one or more instructions, portions, steps, etc. that are non-patient-specific. Likewise, a patient-specific or personalized medical device can include one or more components that are non-patient-specific, and/or can be used with an instrument or tool that is non-patient-specific. Personalized implant designs can be used to manufacture or select patient-specific technologies, including medical devices, instruments, and/or surgical kits. For example, a personalized surgical kit can include one or more patient-specific devices, patient-specific instruments, non-patient-specific technology (e.g., standard instruments, devices, etc.), instructions for use, patient-specific treatment plan information, or a combination thereof.

The system 100 includes a client computing device 102, which can be a user device, such as a smart phone, mobile device, laptop, desktop, personal computer, tablet, phablet, or other such devices known in the art. As discussed further herein, the client computing device 102 can include one or more processors, and memory storing instructions executable by the one or more processors to perform the methods described herein. The client computing device 102 can be associated with a healthcare provider (e.g., a surgeon, healthcare administrator, hospital system, etc.) that is treating the patient. Although FIG. 1 illustrates a single client computing device 102, in alternative embodiments, the client computing device 102 can instead be implemented as a client computing system encompassing a plurality of computing devices, such that the operations described herein with respect to the client computing device 102 can instead be performed by the client computing system and/or the plurality of client computing devices.

The client computing device 102 is configured to receive a patient data set 108 associated with a patient to be treated. The patient data set 108 can include data representative of the patient's demographics and health. The patient demographic information can include patient identification number (ID), name, age, sex, height, weight, body mass index (BMI), occupation, activity level, health rating, or the like. The health information can include data representative of the patient's condition, anatomy, pathology, symptoms, comorbidities, medical history, preferences, and/or any other information or parameters relevant to the patient. Such data can include previous surgical intervention data, previous treatment outcome data, progress data (e.g., surgeon notes), patient feedback (e.g., feedback acquired using quality of life questionnaires, surveys), clinical data, vital signs, medication information, allergies, or the like. In some embodiments, the patient data set can also include image data, such as camera images, Magnetic Resonance Imaging (MRI) images, ultrasound images, Computerized Aided Tomography (CAT) scan images, Positron Emission Tomography (PET) images, X-Ray images, and the like.

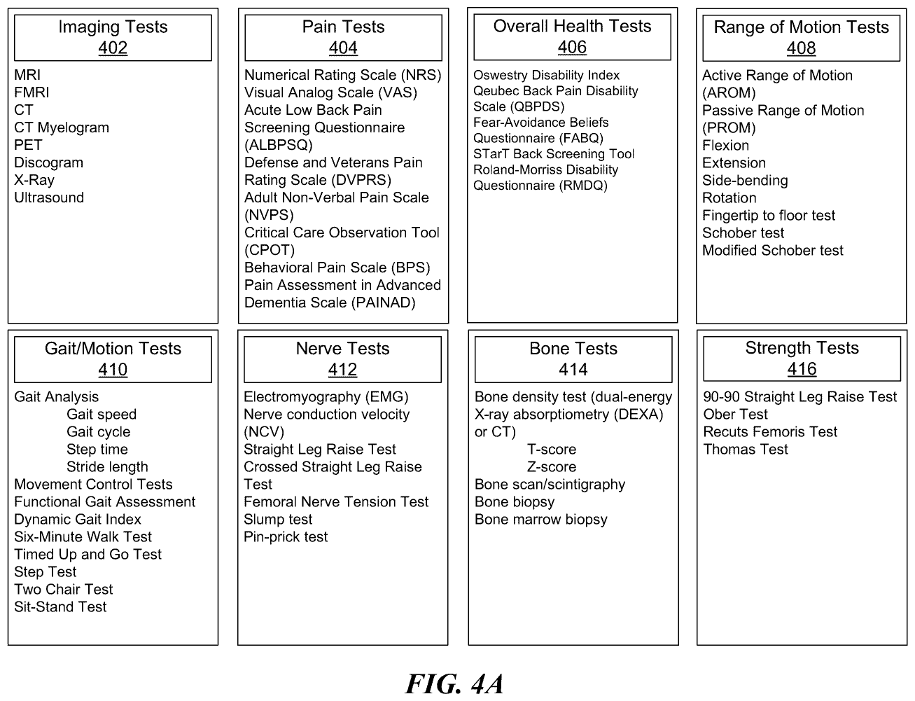

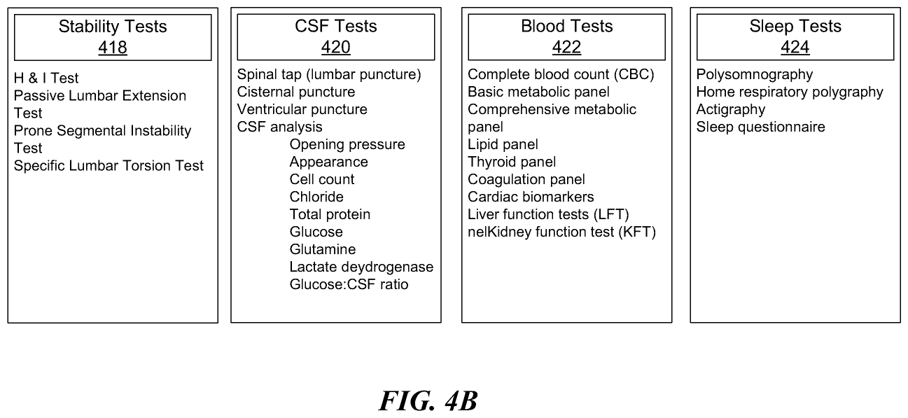

The client computing device 102 is also configured to receive a diagnostic information data set 109 associated with the patient to be treated. The diagnostic information data set 109 can include results from various diagnostic tests performed on the patient. Examples of diagnostic tests include, but are not limited to, imaging tests, pain tests/questionnaires, overall health tests/questionnaires, range of motion tests, gait and other motion tests, nerve tests, bone tests, strength tests, stability tests, cerebral spinal fluid tests, blood tests, sleep tests, and other miscellaneous tests. Additional details of example diagnostic tests for collecting the diagnostic information data set 109 are described below with reference to FIGS. 4A and 4B.

The client computing device 102 is also configured to enable a user (e.g., a surgeon) to review a diagnostic plan and/or a surgical plan for a patient to be treated. In particular, the client computing device 102 can include a treatment plan review software module 123 (“the review module 123”). The review module 123 can comprise computer-executable instructions for generating, displaying, and/or implementing a treatment plan review program or platform 125 (“the review program 125”) that facilitates surgeon or user review of one or more patient-specific diagnostic plans and/or patient-specific surgical plans via the client computing device 102.

The review module 123 can be stored in the form of computer-readable or computer-executable instructions on a memory (not shown) of the client computing device 102. In other embodiments, the review module 123 can be stored remotely from the client computing device 102 (e.g., in the cloud or at a remote server) and implemented on the client computing device 102 via a remote (e.g., wireless) connection. In yet other embodiments, some of the review module 123 can be stored locally at the client computing device 102 while other aspects of the review module 123 can be store remotely.

The client computing device 102 is operably connected via a communication network 104 to a server 106, thus allowing for data transfer between the client computing device 102 and the server 106. The communication network 104 may be a wired and/or a wireless network. The communication network 104, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 106, which may also be referred to as a “treatment assistance network” or “prescriptive analytics network,” can include one or more computing devices and/or systems. As discussed further herein, the server 106 can include one or more processors, and memory storing instructions executable by the one or more processors to perform some or all of the methods described herein. In some embodiments, the server 106 is implemented as a distributed “cloud” computing system or facility across any suitable combination of hardware and/or virtual computing resources.

The client computing device 102 and server 106 can individually or collectively perform some or all of the various methods described herein for providing patient-specific medical care. For example, some or all of the steps of the methods described herein can be performed by the client computing device 102 alone, the server 106 alone, or a combination of the client computing device 102 and the server 106. Thus, although certain operations are described herein with respect to the server 106, it shall be appreciated that these operations can also be performed by the client computing device 102, and vice-versa, unless the context clearly dictates otherwise.

The server 106 includes at least one database 110 configured to store reference data useful for the diagnostic and treatment planning methods described herein. The reference data can include historical and/or clinical data from the same or other patients, data collected from prior diagnostic tests, surgeries, and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like. In some embodiments, the reference data includes third-party data collected from external sources (e.g., remote servers, databases, websites, journals, articles, social media networks, regulatory databases, insurance databases, etc.).

In some embodiments, the database 110 includes a plurality of reference patient data sets, each patient reference data set associated with a corresponding reference patient. For example, the reference patient can be a patient that previously received treatment or is currently receiving treatment. Each reference patient data set can include patient demographic information, patient health information, and patient diagnostic information. Accordingly, each reference patient data set can include data representative of the reference patient's condition, anatomy, pathology, medical history, disease progression, preferences, and/or any other information or parameters relevant to the reference patient, such as any of the data described herein with respect to the patient data set 108 and the diagnostic information data set 109. In some embodiments, the reference patient data set includes pre-operative data, intra-operative data, and/or post-operative data. For example, a reference patient data set can include data representing one or more of patient ID, age, gender, BMI, lumbar lordosis, Cobb angle(s), pelvic incidence, disc height, segment flexibility, bone quality, rotational displacement, and/or treatment level of the spine. As another example, a reference patient data set can include treatment data regarding at least one surgical procedure performed on the reference patient, such as descriptions of surgical procedures or interventions (e.g., surgical approaches, bony resections, surgical maneuvers, corrective maneuvers, placement of implants or other devices). In some embodiments, the treatment data includes medical device design data for at least one medical device used to treat the reference patient, such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). In yet another example, a reference patient data set can include outcome data representing an outcome of the treatment of the reference patient, such as corrected anatomical metrics, presence of fusion, HRQL, pain level, activity level, return to work, complications, recovery times, efficacy, mortality, and/or follow-up surgeries.

In some embodiments, the server 106 receives at least some of the reference patient data sets from a plurality of healthcare provider computing systems (e.g., systems 112a-112c, collectively 112). The server 106 can be connected to the healthcare provider computing systems 112 via one or more communication networks (not shown). Each healthcare provider computing system 112 can be associated with a corresponding healthcare provider (e.g., physician, surgeon, medical clinic, hospital, healthcare network, etc.). Each healthcare provider computing system 112 can include at least one reference patient data set (e.g., reference patient data sets 114a-114c, collectively 114) associated with reference patients treated by the corresponding healthcare provider. The reference patient data sets 114 can include, for example, electronic medical records, electronic health records, biomedical data sets, etc. The reference patient data sets 114 can be received by the server 106 from the healthcare provider computing systems 112 and can be reformatted into different formats for storage in the database 110. Optionally, the reference patient data sets 114 can be processed (e.g., cleaned) to ensure that the represented patient parameters are likely to be useful in the treatment planning methods described herein.

In some embodiments, the server 106 receives at least some of the reference patient data sets from a third-party platform or database. The server 160 can analyze collected reference patient data and group the collected reference patient data into subsets. The server 106 can then assign confidence scores to respective subsets of data. This allows for grouping and confidence-based ranking of grouped data. The database 115 can store collected third-party data, confidence scores, confidence scoring routines, and the like.

As described in further detail herein, the server 106 can be configured with one or more algorithms that generate patient-specific diagnostic plans and/or patient-specific surgical plan data (e.g., treatment procedures, target anatomical corrections, medical devices, etc.) based on the reference data. In some embodiments, the patient-specific data is generated based on correlations between the patient data set 108 and the reference data. Optionally, the server 106 can predict outcomes, including recovery times, efficacy based on clinical end points, likelihood of success, predicted mortality, predicted related follow-up surgeries, or the like. In some embodiments, the server 106 can continuously or periodically analyze patient data (including patient data obtained during the patient stay) to determine near real-time or real-time risk scores, mortality prediction, etc.

In some embodiments, the server 106 includes one or more modules for performing one or more steps of the patient-specific treatment planning methods described herein. For example, in the depicted embodiment, the server 106 includes a diagnostic planning module 115, a diagnostic-driven anatomical modeling module 116, a data analysis module 117, a treatment planning module 118, a disease progression module 120, and an intervention timing module 121. The server 106 can also include additional modules, such as a data similarity module, a third-party collection platform, or the like. In alternative embodiments, one or more of these modules may be combined with each other, or may be omitted. Thus, although certain operations are described herein with respect to a particular module or modules, this is not intended to be limiting, and such operations can be performed by a different module or modules in alternative embodiments.

The diagnostic planning module 115 is configured to analyze the received patient data set 108 to determine diagnostic information expected to be useful in determining a patient-specific treatment plan for the patient. Accordingly, the diagnostic planning module 115 may include software that analyzes the patient data set 108 to (a) determine diagnostic information expected to be useful based on the patient's health and demographics, and (b) identify one or more diagnostic tests designed to collect the diagnostic information. The diagnostic planning module 115 can also identify the one or more diagnostic tests based on other factors, such as diagnostic equipment available at the healthcare provider location treating the patient. Optionally, the diagnostic planning module 115 may identify the diagnostic information and/or diagnostic tests using any of the artificial intelligence architectures or other computing architectures described below with reference to the treatment planning module 118. The diagnostic tests identified by the diagnostic planning module 115 can include but are not limited to, different types of imaging tests, pain tests/questionnaires, overall health tests/questionnaires, range of motion tests, gait and other motion tests, nerve tests, bone tests, strength tests, stability tests, cerebral spinal fluid tests, blood tests, sleep tests, and other miscellaneous tests. Additional details of example diagnostic tests are described below with reference to FIGS. 4A and 4B.

The diagnostic planning module 115 can generate a patient-specific diagnostic plan based on its analysis. The patient-specific diagnostic plan (also referred to as a “diagnostic plan”) can include the one or more diagnostic tests as well as a recommended timing for performing the one or more diagnostic tests and/or instructions for performing the one or more diagnostic tests. The diagnostic plan can be a digital report that can be transmitted to the client computing device 102 (e.g., via the communication network 104) for user review and execution. After the diagnostic plan is executed, the results from the diagnostic tests can be inputted or otherwise received at the client computing device in the form of the diagnostic information data set 109. The diagnostic information data set 109 can then be uploaded or otherwise transmitted to the server 106, e.g., via the communication network (similar to the patient data set 108 being uploaded to the server 106).

The diagnostic-driven anatomical modeling module 116 (the “modeling module 116”) can generate one or more virtual models based on the diagnostic information data set 109 or other diagnostic data (e.g., diagnostic data collected in real time). The virtual models can then be used, for example, to simulate pre-operative mobility, predict mobility (e.g., intra-operative mobility, predict post-operative mobility, etc.), predict treatment outcomes, etc. In some embodiments, the diagnostic information data set 109 or other diagnostic data can be used by the modeling module 116 to create an anatomical model of the person's spine. The anatomical model can be created using an image capture processes, kinematic analysis, or the like. In motion capture embodiments, a person's movement can be recorded using one or more cameras, sensors placed on different parts of the patient's body, and/or tracking equipment. For example, sensors can record movements and transmit movement data the modeling module 116, which analyzes the movement data and creates a virtual model of the person's movements. In some embodiments, a virtual anatomical model can be generated based on image data, and anatomical elements of the virtual model can be moved to match the captured image data. A display 122 can display the patient's movement to compare simulations to other diagnostic data.

To create a diagnostic-driven anatomical model, captured motion data (e.g., image data, videos, sensor data, accelerometer data, gyroscope data, positional data, etc., which may optionally be received as part of the diagnostic information data set 109) can be used to determine the biomechanics, position, orientation, and/or movement of body parts of the patient during, for example, one or more phases of activity. Example activities include walking, running, lifting, jumping, swimming, golfing, biking, etc. This data can then be mapped to an anatomical model of the patient. The motion data can be mapped to the anatomical data using one or more segmentation routines (e.g., segmentation routines for segmenting patient images), identification routines (e.g., identification of anatomic elements, such as vertebral bodies, organs, etc.), mapping routines (e.g., routines for mapping anatomic elements in imaged body parts to anatomic elements in virtual models), kinematic databases (e.g., reference kinematic relationships, biomechanics, etc.), etc. This will allow for a detailed analysis of the pathology and/or movements of, for example, body parts (e.g., legs, arms, etc.), each vertebra, spinal segment, and/or entire spinal column during an activity, such as sitting, jumping, or walking. The resulting diagnostic-driven anatomical model can be used to evaluate one or more of biomechanics, spinal misalignment, spinal health, and/or identify any issues or abnormalities of the spine. Abnormalities can be identified for viewing on the display 122. As described in detail below, the diagnostic-driven anatomical model can also be used to, for example, evaluate patient health, design patient specific customized treatment plans, patient-specific devices (e.g., prosthetics, implants, rehabilitation devices, etc.), rehabilitation programs, and/or patient specific treatments. The simulations can be performed using a pre-operative diagnostic-driven anatomical model representing the patient's pre-operative pathology, a post-operative diagnostic-driven anatomical model representing the patient's post-operative anatomy (e.g., anatomy representing a corrected pathology of the patient, etc.), or the like.

In some embodiments, a virtual model of patient anatomy (e.g., the diagnostic-driven anatomical model) can be validated by comparing simulated motion data associated with the virtual model with collected patient motion data (e.g., the “raw” data or “input” data). This can be performed manually (e.g., by a surgeon or other user) or via a “checker” submodule of the diagnostic-driven anatomical modeling module 116. The surgeon or submodule can confirm accuracy of the virtual model when the virtual model data generally matches the patient motion data. In such embodiments, the virtual model data can be overlaid onto videos and/or images of the patient. The modeling module 116 can store matching settings for determining when, for example, the motion data from the virtual model matches the patient motion data with a threshold confidence score. In some embodiments, a virtual model can be validated by comparing measurements from a virtual model with collected patient measurements. The modeling module 116 can store matching measurement settings for determining when, for example, the measurements from the virtual model matches the patient measurements within a threshold confidence score. The modeling module 116 can repeatedly modify the virtual model and/or surgical plan until at least one validated virtual model is obtained.

Virtual models such as the diagnostic-driven anatomical model can be created using computer aided design (CAD) software to simulate, for example, biomechanics, surgical procedures, and outcomes. The virtual model can be generated, modified, and/or validated based on model simulation data. Model simulation data can include, for example, measurements of the virtual spine model, such as pre-operative measurements, intra-operative measurements, and/or post-operative measurements. Example measurements include changes in angles and distances between anatomic elements, such as vertebrae as well as any other relevant parameters. A comparison of simulation data to diagnostic data can be performed using one or more comparison algorithms to detect differences and similarities between the two datasets. As described in detail below, a recommended surgical procedure can be identified based on the simulated data matching the diagnostic data and meeting one or more desired surgical outcomes. The surgical outcomes can be inputted by a healthcare provider or determined using machine learning algorithms. A surgical plan can then be generated based on the recommended surgical procedure.

In some embodiments, a set of anatomical positions can be modeled using a diagnostic-driven anatomical model. Biomechanical properties can be measured using the virtual model at each of the anatomical positions. In some embodiments, the diagnostic-driven anatomical models can therefore be referred to as biomechanical virtual models. The anatomical positions include, for example, a supine position, prone position, standing position, under flexion position, under extension position, neutral position, or side-bending position. The positions can be selected based on, for example, available diagnostic data (e.g., measurements of the patient in the supine position, prone position, standing position, under flexion position, under extension position, neutral position, or side-bending position) and/or and modeling capabilities of the modeling module 116.

In some embodiments, a virtual model representing the patient's spine can be generated based on the image data. The virtual model can include, for example, anatomic elements that can be moved to modify the virtual model. For example, virtual models of a spine can include movable vertebral bodies or vertebrae. A user can reposition the vertebrae to provide a targeted correction. Virtual models can include other types of anatomical elements capable of being repositioned relative to each other, a reference point, or the like. A biomechanical virtual model can be generated based on the virtual model. One or more metrics can be determined based on the biomechanical virtual model. In some implementations, the biomechanical virtual model can be viewed by a user. To treat a patient's spine, the biomechanical virtual model can be based on the virtual model of the patient. One or more spinal metrics can be extrapolated from the biomechanical model for viewing by the user. For example, the biomechanical model can include simulations for flexion, extension, rotation and lateral flexion, or other information of the spine. The biomechanical model can be updated based on user inputted changes to spinal metrics. If the user modifies disc height at one more levels, the biomechanical model can be updated to show how the modifications impact the simulations. This allows the user to repeatedly adjust biomechanical models to evaluate stability, complex motions, or other features of the patient's spine. The biomechanical models may also include motion parameters associated with the patient's anatomy for simulating movement. The motion parameters can be derived from the obtained diagnostic data. The system can assign the motion parameters to the virtual model to generate all these a portion of biomechanical virtual model. For example, the biomechanical virtual model can include the virtual model with motion parameters (e.g., positional relationships, motion parameters, constraints, parametric parameters, etc.) assigned to anatomical elements the virtual model. The motion parameters can be derived from one or more gait tests, motion tests, spinal stability tests, and/or other tests disclosed herein.

In some embodiments, one or more implants can be designed using the biomechanical virtual model. Spinal implants can be designed to provide a desired amount of movement or repositioning of anatomy of the postoperative biomechanical virtual model. Artificial spinal discs can be designed based on simulations generated using the biomechanical virtual model including the designed spinal disc. This allows a user to understand how an artificial spinal disc will perform under various conditions. An implant design can therefore be generated based on the virtual model, and can include information from the biomechanical virtual model. The information can include images of the virtual model, videos the biomechanical virtual model showing motion data, including pre-operative motion data, intra-operative motion data, and/or post-operative motion data. Additional details regarding designing implants based on virtual models are described below.

In some embodiments, the system 100 can determine one or more candidate modifications for virtual models based on patient images. The system 100 can determine additional diagnostic data needed to analyze the candidate modifications. The system 100 can then send a request to a user for the needed diagnostic data. The system 100 can retrieve additional information needed to analyze any number of candidate modifications of a virtual model, biomechanical virtual model, or other model discussed herein. The system can rank candidate modifications for the virtual model and then generate an optimal virtual model based on a user selected candidate modification, a highest ranked candidate modification, etc.

The system 100 can also modify models based on an orientation of the patient's spine when data was obtained. The modifications can compensate for the state of the patient's spine when the image data was obtained. For example, the system can determine modifications to the virtual model based on an orientation of the patient's spine when the image data was captured. The modifications can compensate for pathology of patient's spine when the image data was captured.

In some embodiments, the system 100 can import one or more virtual models generated via a third-party program (e.g., a computer-aided design platform or application). In such embodiments, the computing deice 102 can retrieve the one or more virtual models from the third-party program, and the modeling module 116 can validate the third-party model for planning (e.g., diagnostic planning, treatment planning, etc.). For example, the third-party model can be validated by authenticating the third-party data source, translating the third-party model into a readable digital format, modifying the third-party virtual model for importation into an existing model, confirming that the imported third-party virtual model is compatible with other model elements, and/or importing all or a portion of the third-party virtual model and associated data (e.g., diagnostic data or metrics, spinopelvic metrics or parameters). The imported third-party model can be, for example, a two-dimensional (2D), three-dimensional (3D) model, or other model of one or more anatomical elements, implants, navigation equipment, and/or instruments.

In some embodiments, the system 100 combines multiple virtual models to generate a multi-source model. The multiple virtual models can include models generated by the modelling module 116 and/or models generated from one or more third-party platforms. In such embodiments, the modeling module 116 can analyze, score, rank, and/or modify the multi-source model to, for example, match patient diagnostic data, patient images, predictions, surgical plans, etc. The models include standard elements/models modifiable (e.g., scaled, sized, or altered to match patient data) based on patient data, patient-specific models with topology matching patient topology, etc. For example, the modeling module 116 can combine one or more standard elements/models of anatomy, patient-specific models, models of implants, and other models to generate multi-source models. In spine embodiments, the standard elements/models can be, for example, standard or generic vertebrae positioned to represent the patient's spine. The client computing device 102 can retrieve the standard elements/models from a library or database. In some embodiments, the client computing device 102 selects standard element/model based on the patient's age, gender, body type, etc. The elements/model can be positioned relative to one another based on patient measurements to model positional relationships between anatomic elements, thereby creating a patient-specific model utilizing standard or generic anatomical elements. A virtual location model of anatomy can be displayed for viewing by a user. In some embodiments, a model includes both standard elements/models and patient-specific elements/models. The standard elements/models can be used when an insufficient amount of information is available to generate patient-specific elements/models.

The multi-source models can be location models, topology models, and location-topology models and can be, for example, updated or modified using the patient data 108 and/or the diagnostic data 109. The location models can represent the anatomical positions of the patient anatomy and can include, for example, standard anatomical elements, patient-specific anatomical elements, and/or features disclosed herein. The topology models can represent the topology of the patient's anatomy. When an insufficient amount of information is available to generate topology (e.g., an entire vertebral endplate), the modeling module 116 can generate approximated topologies using standard topologies, ML modules, etc. The location-topology models can represent both the position and the topology of the patient's anatomy. In some embodiments, the multi-source models can be 2D models, 3D models, etc. The client computing device 102 can therefore include import and export modules, e.g., from or to various third-party design platforms. The client computing device 102 and/or user can select metrics and/or parameters for the multi-source models. If additional diagnostic data is available, models and simulations can be modified or updated based on the newly available diagnostic data.

The data analysis module 117 is configured with one or more algorithms for identifying a subset of reference data from the database 110 that is likely to be useful in developing the patient-specific diagnostic plan and/or the patient-specific treatment plan. For example, the data analysis module 117 can compare patient-specific data (e.g., the patient data set 108 and/or the diagnostic information data set 109 received from the client computing device 102; collectively referred to as “the data sets 108/109”) to the reference data from the database 110 (e.g., the reference patient data sets) to identify similar data (e.g., one or more similar data sets in the reference data sets). In the context of determining a patient-specific diagnostic plan, the comparison can be based on patient demographics and health data. In the context of determining a patient-specific treatment plan, the comparison can be based on patient demographics, patient health data, and any diagnostic information collected as part of the patient-specific diagnostic plan. For example, the comparison can be based on one or more parameters, such as age, sex, height, weight, lumbar lordosis, pelvic incidence, pain rating, and/or treatment levels. The parameter(s) can be used to calculate a similarity score for each reference patient. The similarity score can represent a statistical correlation between the data sets 108/109 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients.

The data analysis module 117 can further be configured with one or more algorithms to select a subset of the reference patient data sets, e.g., based on similarity to the data sets 108/109 and/or treatment outcome of the corresponding reference patient. For example, the data analysis module 117 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, presence of fusion, HRQL, activity level, complications, recovery times, efficacy, mortality, or follow-up surgeries. As described in further detail below, in some embodiments, the data analysis module 117 calculates an outcome score by assigning values to each outcome parameter. A patient can be considered to have a favorable outcome if the outcome score is above, below, or at a specified threshold value.

In some embodiments, the diagnostic planning module 115 can generate one or more recommended diagnostic tests based on analysis conducted by the data analysis module 117. For example, if the data analysis module 117 is unable to identify a suitable subset of reference data for determining a patient-specific surgical plan (e.g., the data analysis module 117 identifies not enough or too many reference patients, identifies reference patients with divergent similarity scores or outcomes, etc.), the diagnostic planning module 115 can determine additional diagnostic tests to be performed to obtain additional diagnostic information. The additional diagnostic information can be used by the data analysis module 117 as additional parameters for identifying the subset of reference data useful in developing the patient-specific treatment plan.

In some embodiments, the data analysis module 117 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 117 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular health-care provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific surgical plan to perform a battlefield surgery can be based on reference patient data from similar battlefield surgeries and/or data sets associated with battlefield surgeries. In another example, the patient-specific surgical plan can be generated based on available robotic surgical systems. The reference patient data sets can be selected based on patients that have been operated on using comparable robotic surgical systems under similar conditions (e.g., size and capabilities of surgical teams, hospital resources, etc.).

The treatment planning module 118 is configured with one or more algorithms to generate at least one surgical plan (e.g., pre-operative plans, intra-operative plans, post-operative plans etc.) based on the output from the data analysis module 117. In some embodiments, the treatment planning module 118 is configured to develop and/or implement at least one predictive model for generating the patient-specific treatment plan, also known as a “prescriptive model.” The predictive model(s) can be developed using clinical knowledge, statistics, machine learning, AI, neural networks, or the like. In some embodiments, the output from the data analysis module 117 is analyzed (e.g., using statistics, machine learning, neural networks, AI) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a surgical plan will produce a favorable outcome for the particular patient. The predictive model(s) can be validated, e.g., by inputting data into the model(s) and comparing the output of the model to the expected output.

In some embodiments, the treatment planning module 118 is configured to generate the surgical plan based on previous treatment data from reference patients. For example, the treatment planning module 118 can receive a selected subset of reference patient data sets and/or similar patient data sets from the data analysis module 117, and determine or identify treatment data from the selected subset. The treatment data can include, for example, treatment procedure data (e.g., surgical procedure or intervention data) and/or medical device design data (e.g. implant design data) that are associated with favorable or desired treatment outcomes for the corresponding patient. The treatment planning module 118 can analyze the treatment procedure data and/or medical device design data to determine an optimal treatment protocol for the patient to be treated. For example, the treatment procedures and/or medical device designs can be assigned values and aggregated to produce a treatment score. The patient-specific surgical plan can be determined by selecting surgical plan(s) based on the score (e.g., higher or highest score; lower or lowest score; score that is above, below, or at a specified threshold value). The personalized patient-specific surgical plan can be based on, at least in part, the patient-specific technologies or patient-specific selected technology.

Alternatively or in combination, the treatment planning module 118 can generate the surgical plan based on correlations between data sets. For example, the treatment planning module 118 can correlate treatment procedure data and/or medical device design data from similar patients with favorable outcomes (e.g., as identified by the data analysis module 117). Correlation analysis can include transforming correlation coefficient values to values or scores. The values/scores can be aggregated, filtered, or otherwise analyzed to determine one or more statistical significances. These correlations can be used to determine surgical procedure(s) and/or medical device design(s) that are optimal or likely to produce a favorable outcome for the patient to be treated.

Alternatively or in combination, the treatment planning module 118 can generate the surgical plan using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 118 generates the surgical plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 110, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training data set can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate a surgical plan, the data sets 108/109 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures and/or medical device designs are likely to produce a favorable outcome for the patient. Based on these calculations, the trained machine learning model(s) can select at least one surgical plan for the patient. In some embodiments, the trained machine learning model(s) can determine candidate procedures (or candidate surgical plans), analyze the candidate procedures, select the candidate surgical plans or portions thereof, score plans, and/or generate surgical plans for the patient. Each surgical plan can be scored (e.g., scored based on favorable outcome, likelihood of outcome, etc.) and ranked according to the score. The trained machine learning model(s) can determine a set of surgical plans that meet selection criteria for plan review by a user. The selection criteria can be based on, for example, regulatory requirements, reimbursement criteria, healthcare/provider expertise, available surgical equipment, manufacturing capabilities, elimination criteria, combinations thereof, or the like. A user can input one or more selection criteria to control the types and/or features of the surgical plans for comparison. In embodiments where multiple trained machine learning models are used, the models can be run sequentially or concurrently to compare outcomes and can be periodically updated using training data sets. The treatment planning module 118 can use one or more of the machine learning models based the model's predicted accuracy score.

The patient-specific surgical plan generated by the treatment planning module 118 can include at least one patient-specific surgical procedure (e.g., a surgical procedure or intervention) and/or at least one patient-specific medical device (e.g., an implant or implant delivery instrument). A patient-specific surgical plan can include an entire surgical procedure or portions thereof. Additionally, one or more patient-specific medical devices can be specifically selected or designed for the corresponding surgical procedure, thus allowing for the various components of the patient-specific technology to be used in combination to treat the patient.

In some embodiments, the patient-specific surgical procedure includes an orthopedic surgery procedure, such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). In some embodiments, the patient-specific treatment procedure includes descriptions of and/or instructions for performing one or more aspects of a patient-specific surgical procedure. For example, the patient-specific surgical procedure can include one or more of a surgical approach, a corrective maneuver, a bony resection, or implant placement.

In some embodiments, the patient-specific medical device design includes a design for an orthopedic implant and/or a design for an instrument for delivering an orthopedic implant. Examples of such implants include, but are not limited to, screws (e.g., bone screws, spinal screws, pedicle screws, facet screws), interbody implant devices (e.g., intervertebral implants), cages, plates, rods, disks, fusion devices, spacers, rods, expandable devices, stents, brackets, ties, scaffolds, fixation device, anchors, nuts, bolts, rivets, connectors, tethers, fasteners, joint replacements, hip implants, or the like. Examples of instruments include, but are not limited to, screw guides, cannulas, ports, catheters, insertion tools, removal tools, awls, drivers, or the like.

A patient-specific medical device design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of a corresponding medical device. For example, a design for an orthopedic implant can include implant shape, size, material, and/or effective stiffness (e.g., lattice density, number of struts, location of struts, etc.). In some embodiments, the generated patient-specific medical device design is a design for an entire device. Alternatively, the generated design can be for one or more components of a device, rather than the entire device.

In some embodiments, the design is for one or more patient-specific device components that can be used with standard, off-the-shelf components. For example, in a spinal surgery, an ALIF kit can include both standard components and patient-specific customized components. In some embodiments, the ALIF kit can include a patient specific interbody device that can be used with standard interfixating screws. In some embodiments, the generated design is for a patient-specific implant that can be used with a standard, off-the-shelf delivery instrument. For example, the implants (e.g., interbody device, screws, screw holders, rods) can be designed and manufactured for the patient, while the instruments for delivering the implants can be standard instruments. This approach allows the components that are implanted to be designed and manufactured based on the patient's anatomy and/or surgeon's preferences to enhance treatment. The patient-specific devices described herein are expected to improve delivery into the patient's body, placement at the treatment site, and/or interaction with the patient's anatomy.

In embodiments in which the patient-specific surgical plan includes a specific surgical procedure to implant a medical device, the treatment planning module 118 can also store various types of implant surgery information, such as implant parameters (e.g., types, dimensions), availability of implants, aspects of a pre-operative plan (e.g., initial implant configuration, detection and measurement of the patient's anatomy, etc.), FDA requirements for implants (e.g., specific implant parameters and/or characteristics for compliance with FDA regulations), or the like. In some embodiments, the treatment planning module 118 can convert the implant surgery information into formats useable for machine-learning based models and algorithms. For example, the implant surgery information can be tagged with particular identifiers for formulas or can be converted into numerical representations suitable for supplying to the trained machine learning model(s). The treatment planning module 118 can also store information regarding the patient's anatomy, such as two- or three-dimensional images or models of the anatomy, and/or information regarding the biology, geometry, and/or mechanical properties of the anatomy. The anatomy information can be used to inform implant design and/or placement.

In some embodiments, the system 100 can import one or more plans (e.g., pre-operative plans, intra-operative plans, post-operative plans, etc.) from a third-party data source, e.g., in addition to or in lieu of generating a surgical plan using the treatment planning module 118. In such embodiments, the client computing device 102 can receive multiple plans (e.g., diagnostic plans, treatment plans, etc.) and the treatment planning module 118 can integrate the plans together to generate a single multi-source treatment plan. For example, client computing device 102 can select a set of metrics from a first treatment plan and a second set of metrics from a second treatment plan. A multi-source treatment plan and a virtual anatomical model can be generated to achieve both the first and second sets of metrics. For example, the treating planning module 118 can generate a multi-treatment plan based on the multiple individual treatment plans, such as an anterior fusion plan (e.g., a lumbar fusion plan for implanting one or more cages) and a posterior fusion plan (e.g., a fusion plan for implanting one or more rods). The multi-treatment plan can simulate outcomes associated with each treatment, spine pathology, and the like. The multi-treatment plan can be synchronized to update information (e.g., metrics, types of visual images, etc.) selected by, for example, a physician, healthcare provider, the client computing device 102, machine learning module, or the like. The metrics and/or parameters can be from different models and/or treatment plans. In some embodiments, the multi-treatment plan can be updated based on modification(s) to a linked third-party source that provided a treatment plan.

The disease progression module 120 can be used to analyze, predict, and/or model disease progression for a particular patient. As described in detail below, the disease progression module 120 can estimate the rate of disease progression for the patient under a variety of different circumstances, including (a) if no surgical intervention occurs, and (b) if one or more surgical plans (e.g., surgical procedures identified by the treatment planning module 118) are performed. The disease progression module 120 can therefore include an algorithm, machine learning model, or other software analytical tool for predicting disease progression in a particular patient.

In some embodiments, the disease progression module 120 includes a machine learning model or other software module that can be trained based off a plurality of reference patient data sets that includes, in addition to the patient data described above, disease progression metrics for each of the reference patients. The progression metrics can include measurements for disease metrics over a period of time. Suitable metrics may include spinopelvic parameters (e.g., lumbar lordosis, pelvic tilt, sagittal vertical axis (SVA), cobb angel, coronal offset, etc.), disability scores, functional ability scores, flexibility scores, VAS pain scores, or the like. The progression of the metrics for each reference patient can be correlated to other patient information for the specific reference patient (e.g., age, sex, height, weight, activity level, diet, etc.). The disease metrics can include values over a period of time. For example, the reference patient data may include values of disease metrics on a daily, weekly, monthly, bi-monthly, yearly, or other basis. By measuring the metrics over a period of time, changes in the values of the metrics can be tracked as an estimate of disease progression and correlated to other patient data.

In some embodiments, the disease progression module 120 can therefore estimate the rate of disease progression for a particular patient. The progression may be estimated by providing estimated changes in one or more disease metrics over a period of time (e.g., X % increase in a disease metric per year). The rate can be constant (e.g., 5% increase in pelvic tilt per year) or variable (e.g., 5% increase in pelvic tilt for a first year, 10% increase in pelvic tilt for a second year, etc.). In some embodiments, the estimated rate of progression can be transmitted to a surgeon or other healthcare provider as part of a surgical plan, as described in greater detail below.

As a non-limiting example, a particular patient who is a fifty-five-year-old male may have a SVA value of 6 mm. The disease progression module 120 can analyze patient reference data sets to identify disease progression for individual reference patients having one or more similarities with the particular patient (e.g., individual patients of the reference patients who have an SVA value of about 6 mm and are approximately the same age, weight, height, and/or sex of the patient). Based on this analysis, the disease progression module 120 can predict the rate of disease progression if no surgical intervention occurs (e.g., the patient's VAS pain scores may increase 5%, 10%, or 15% annually if no surgical intervention occurs, the SVA value may continue to increase by 5% annually if no surgical intervention occurs, etc.).

The surgical treatment plans and/or associated patient-specific implants described herein can also be at least partially based on the estimated rates of disease progression, enabling the modeling of different outcomes over a desired period of times. Additionally, the models/simulations can account for any number of additional diseases or conditions to predict the patient's overall health, mobility, or the like. These additional diseases or conditions can, in combination with other patient health factors (e.g., height, weight, age, activity level, etc.) be used to generate a patient health score reflecting the overall health of the patient. The patient health score can be displayed for surgeon review and/or incorporated into the estimation of disease progression. Accordingly, the present technology can generate one or more virtual simulations of the predicted disease progression to demonstrate how the patient's anatomy is predicted to change over time. Physician input can be used to generate or modify the virtual simulation(s). The present technology can generate one or more post-treatment virtual simulations based on the received physician input for review by the healthcare provider, patient, etc.

In some embodiments, the present technology can also predict, model, and/or simulate disease progression based on one or more potential surgical plans. For example, the disease progression module 120 may simulate what a patient's anatomy and/or spinal metrics may be 1, 2, 5, or 10 years post-surgery for several different surgical plans. The simulations may also incorporate non-surgical factors, such as patient age, height, weight, sex, activity level, other health conditions, or the like, as previously described. The system and/or a surgeon can use the disease progression to aid in selecting which surgical plan provides the best long-term efficacy, as described below. These simulations can also be used to determine patient-specific corrections that compensate for the projected diseases progression.

Accordingly, in some embodiments, multiple disease progression models (e.g., two, three, four, five, six, or more) are simulated to provide disease progression data for several different surgical plans. For example, the disease progression module can generate models that predict post-surgical disease progression for each of three different surgical plans. A surgeon or other healthcare provider can review the disease progression models and, based on the review, select which of the three surgical plans is likely to provide the patient with the best long-term outcome.

Based off of the modeled disease progression, the systems and methods described herein can also (i) identify a recommended time for surgical intervention, and/or (ii) identify a recommended type of surgical procedure for the patient. In some embodiments, the present technology therefore includes an intervention timing module 121 that includes an algorithm, machine learning model, or other software analytical tool for determining the optimal time for surgical intervention in a particular patient. This can be done, for example, by analyzing patient reference data that includes (i) pre-operative disease progression metrics for individual reference patients, (ii) disease metrics at the time of surgical intervention for individual reference patients, (iii) post-operative disease progression metrics for individual reference patients, and/or (iv) scored surgical outcomes for individual reference patients. The intervention timing module 121 can compare the disease metrics for a particular patient to the reference patient data sets to determine, for similar patients, the point of disease progression at which surgical intervention produced the most favorable outcomes.

As a non-limiting example, the reference patient data sets may include data associated with reference patients' sagittal vertical axis. The data can include (i) sagittal vertical axis values for individual patients over a period of time before surgical intervention (e.g., how fast and to what degree the sagittal vertical axis value changed), (ii) sagittal vertical axis of the individual patients at the time of surgical intervention, (iii) the change in sagittal vertical axis after surgical intervention, and (iv) the degree to which the surgical intervention was successful (e.g., based on pain, quality of life, or other factors). Based on the foregoing data, the intervention timing module 121 can, based on a particular patient's sagittal vertical axis value, identify at which point surgical intervention will have the highest likelihood of producing the most favorable outcome. Of course, the foregoing metric is provided by way of example only, and the intervention timing module 121 can incorporate other metrics (e.g., lumbar lordosis, pelvic tilt, sagittal vertical axis, cobb angel, coronal offset, disability scores, functional ability scores, flexibility scores, VAS pain scores) instead of or in combination with sagittal vertical axis to predict the time at which surgical intervention has the highest probability of providing a favorable outcome for the particular patient.

The intervention timing module 121 may also incorporate one or more mathematical rules based on value thresholds for various disease metrics. For example, the intervention timing module 121 may indicate surgical intervention is necessary if one or more disease metrics exceed a predetermined threshold or meet some other criteria. Representative thresholds that indicate surgical intervention may be necessary include SVA values greater than 7 mm, a mismatch between lumbar lordosis and pelvic incidence greater than 10 degrees, a cobb angle of greater than 10 degrees, and/or a combination of cobb angle and LL/PI mismatch greater than 20 degrees. Of course, other threshold values and metrics can be used; the foregoing are provided as examples only. In some embodiments, the foregoing rules can be tailored to specific patient populations (e.g., for males over 50 years of age, an SVA value greater than 7 mm indicates the need for surgical intervention). If a particular patient does not exceed the thresholds indicating surgical intervention is recommended, the intervention timing module 121 may provide an estimate for when the patient's metrics will exceed one or more thresholds, thereby providing the patient with an estimate of when surgical intervention may become recommended.

In some embodiments, the treatment planning module 118 identifies one or more types of surgical procedures for the patient based at least in part on the disease progression of the patient determined using the disease progression module 120 and/or the intervention timing module 121. The treatment planning module 118 may also incorporate one or more mathematical rules for identifying surgical procedures. As a non-limiting example, if a LL/PI mismatch is between 10 and 20 degrees, the treatment planning module 118 may recommend an anterior fusion surgery, but if the LL/PI mismatch is greater than 20 degrees, the treatment planning module may recommend both anterior and posterior fusion surgery. As another non-limiting example, if a SVA value is between 7 mm and 15 mm, the treatment planning module may recommend posterior fusion surgery, but if the SVA is above 15 mm, the treatment planning module may recommend both posterior fusion surgery and anterior fusion surgery. Of course, other rules can be used; the foregoing are provided as examples only.

Without being bound by theory, incorporating disease progression modeling into the patient-specific surgical plans described herein may even further increase the effectiveness of the procedures and/or provide a surgeon more data by which to evaluate various surgical plans. For example, in many cases it may be disadvantageous to operate after a patient's disease progresses to an irreversible or unstable state. However, it may also be disadvantageous to operate too early, such as before the patient's disease is causing symptoms and/or if the patient's disease may not progress further. The disease progression module 120 and/or the intervention timing module 121 can therefore help identify the window of time during which surgical intervention in a particular patient has the highest probability of providing a favorable outcome for the patient.

The surgical plan(s) generated by the treatment planning module 118 can be transmitted via the communication network 104 to the client computing device 102 for output to a user (e.g., clinician, surgeon, healthcare provider, patient). In some embodiments, the client computing device 102 includes or is operably coupled to a display 122 for outputting the treatment plan(s). The display 122 can include a graphical user interface (GUI) for visually depicting various aspects of the surgical plan(s). For example, the display 122 can show various aspects of a surgical procedure to be performed on the patient, such as the surgical approach, treatment levels, corrective maneuvers, tissue resection, and/or implant placement. To facilitate visualization, the surgical plan can include a virtual model of the surgical procedure that can be displayed via the display 122.

The display 122 may also display additional aspects of the surgical plan, such as predicted post-operative patient metrics, predicted disease progression metrics associated with the identified surgical procedure, etc. As another example, the display 122 can show a design for a medical device to be implanted in the patient in accordance with the transmitted surgical plan, such as a two- or three-dimensional model of the device design. The display 122 can also show patient information, such as two- or three-dimensional images or models of the patient's anatomy where the surgical procedure is to be performed and/or where the device is to be implanted. The client computing device 102 can further include one or more user input devices (not shown) allowing the user to modify, select, approve, and/or reject the displayed treatment plan(s).

In some embodiments, one or more aspects of the surgical plan are displayed using the surgical plan review program 125. For example, the review program 125, which may be implemented as a mobile phone application, a computer application, or the like, can display (e.g., via the display 122) one or more aspects of the diagnostic plan (e.g., instructions for performing one or more specific diagnostic tests) and/or the surgical plan (e.g., a surgical procedure, a virtual model of patient anatomy, an implant, etc.). The review program 125 may provide an interactive interface that further enables a surgeon to select between different patients, select between different diagnostic plans and/or surgical plans for the same patient, compare diagnostic and/or surgical plans for the same patient, review the status of a diagnostic and/or surgical plan, provide feedback on a proposed diagnostic and/or surgical plan, accept a diagnostic and/or surgical plan, reject a diagnostic and/or surgical plan, etc. The review program 125 may further enable a surgeon or other user to select between different views of a virtual model of patient anatomy (e.g., the diagnostic-driven anatomical model), and/or different views of a patient-specific implant to be used in the surgical plan.

In some embodiments, the review program 125 may provide an interactive interface that enables a surgeon or other user to select utilized diagnostic data, mark diagnostic tests as complete, provide feedback on diagnostic tests, or the like. In some embodiments, a surgeon or other user can validate a virtual model of patient anatomy (e.g., the diagnostic-driven anatomical model) using the review program 125 by comparing simulated motion data associated with the virtual model with collected patient motion data (e.g., the “raw” data or “input” data), similar to described above with reference to the modeling module 116. Thus, in some embodiments the review program 125 enables the surgeon to independently confirm the accuracy of the virtual model when the virtual model data generally matches the patient motion data. Similar to the “checker” submodule described above, in such embodiments the virtual model data can be overlaid onto videos and/or images of the patient for surgeon review.

In some embodiments, the medical device design(s) generated by the treatment planning module 118 can be transmitted from the client computing device 102 and/or server 106 to a manufacturing system 124 for manufacturing a corresponding medical device. The manufacturing system 124 can be located on site or off site. On-site manufacturing can reduce the number of sessions with a patient and/or the time to be able to perform the surgery whereas off-site manufacturing can be useful make the complex devices. Off-site manufacturing facilities can have specialized manufacturing equipment. In some embodiments, more complicated device components can be manufactured off site, while simpler device components can be manufactured on site.

Various types of manufacturing systems are suitable for use in accordance with the embodiments herein. For example, the manufacturing system 124 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 124 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 124 can manufacture one or more patient-specific medical devices based on fabrication instructions or data (e.g., CAD data, 3D data, digital blueprints, stereolithography data, or other data suitable for the various manufacturing technologies described herein). Different components of the system 100 can generate at least a portion of the manufacturing data used by the manufacturing system 124. The manufacturing data can include, without limitation, fabrication instructions (e.g., programs executable by additive manufacturing equipment, subtractive manufacturing equipment, etc.), 3D data, CAD data (e.g., CAD files), CAM data (e.g., CAM files), path data (e.g., print head paths, tool paths, etc.), material data, tolerance data, surface finish data (e.g., surface roughness data), regulatory data (e.g., FDA requirements, reimbursement data, etc.), or the like. The manufacturing system 124 can analyze the manufacturability of the implant design based on the received manufacturing data. The implant design can be finalized by altering geometries, surfaces, etc. and then generating manufacturing instructions. In some embodiments, the server 106 generates at least a portion of the manufacturing data, which is transmitted to the manufacturing system 124.

The manufacturing system 124 can generate CAM data, print data (e.g., powder bed print data, thermoplastic print data, photo resin data, etc.), or the like and can include additive manufacturing equipment, subtractive manufacturing equipment, thermal processing equipment, or the like. The additive manufacturing equipment can be 3D printers, stereolithography devices, digital light processing devices, fused deposition modeling devices, selective laser sintering devices, selective laser melting devices, electronic beam melting devices, laminated object manufacturing devices, powder bed printers, thermoplastic printers, direct material deposition devices, or inkjet photo resin printers, or like technologies. The subtractive manufacturing equipment can be CNC machines, electrical discharge machines, grinders, laser cutters, water jet machines, manual machines (e.g., milling machines, lathes, etc.), or like technologies. Both additive and subtractive techniques can be used to produce implants with complex geometries, surface finishes, material properties, etc. The generated fabrication instructions can be configured to cause the manufacturing system 124 to manufacture the patient-specific orthopedic implant that matches or is therapeutically the same as the patient-specific design. In some embodiments, the patient-specific medical device can include features, materials, and designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

The surgical plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 118 generates control instructions configured to cause a surgical robot (e.g., robotic surgery systems, navigation systems, etc.) to partially or fully perform a surgical procedure. The control instructions can be transmitted to the robotic apparatus by the client computing device 102 and/or the server 106.

Following the treatment of the patient in accordance with the surgical plan, treatment progress can be monitored over one or more time periods to update the diagnostic planning module 115, the modeling module 116, the data analysis module 117, the treatment planning module 118, the disease progression module 120, and/or the intervention timing module 121. Post-treatment data can be added to the reference data stored in the database 110. The post-treatment data can be used to train machine learning models for developing patient-specific treatment plans, patient-specific medical devices, or combinations thereof.

It shall be appreciated that the components of the system 100 can be configured in many different ways. For example, in alternative embodiments, the database 110, the diagnostic planning module 115, the modeling module 116, the data analysis module 117, the treatment planning module 118, the disease progression module 120, and/or the intervention timing module 121 can be components of the client computing device 102, rather than the server 106. As another example, the database 110, the diagnostic planning module 115, the modeling module 116, the data analysis module 117, the treatment planning module 118, the disease progression module 120, and/or the intervention timing module 121 can be located across a plurality of different servers, computing systems, or other types of cloud-computing resources, rather than at a single server 106 or client computing device 102.

Additionally, in some embodiments, the system 100 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like.

In some embodiments, the system 100 can operate with one or more third party platforms or systems. For example, in some embodiments the client computing device 102 can communicate with a third-party data source such that the third-party data source provides, modifies, replaces, or adjusts one or more aspects of the system. In some embodiments, the client computing device 102 can be periodically or continuously synchronized with third-party planning software, CAD software, navigation software, user device (e.g., smartphone or tablet capturing images of surgery site), robotic surgery system, or the like. In some embodiments, the synchronization can be performed based on one or more synchronization triggers, including modification of data or event (e.g., publication of article(s), planned manufacturing of implant, start of surgery, etc.). In some embodiments, a user can set a schedule (e.g., daily, weekly, monthly, etc.) for synchronization using a mobile application.

Further, the server 106 can include a third-party collection module which can be part of, or incorporated into, a healthcare system and can manage the collection and analysis of third-party data for designing implants and generating healthcare plans. The server 106 can use machine learning to generate data confidence scores for the collected third-party data or other data. The confidence scores can be generated based on a data type basis, and the confidence score can be ranked, weighted, and/or combined to determine an overall confidence score. For example, data-type confidence scores can indicate confidence level based on the type of data, including subjective data, objective data, or the like. A confidence score for high-confidence data, such as patient images (e.g., high-resolution X-rays or MRIs), scorable diagnostic tests, peer-reviewed or authenticated journal articles, or other objective data, can be higher than a confidence score for low-confidence data, such as subjective data in the form of physician notes, non-peer-reviewed articles, pictures of a patient's posture, low-resolution X-rays, etc. U.S. Pat. No. 11,793,577 filed Oct. 24, 2023, and U.S. patent application Ser. No. 18/373,899 filed Sep. 27, 2023, disclose types of data, including image data, usable by the third-party collection module and are incorporated by reference in their entireties.

The third-party collection module can collect and analyze third-party data using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the third-party collection module collects and analyzes third-party data using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in the database 110, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training data set can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set can fluctuate during training, such that ad hoc rules may be used to decide when overfitting has truly begun. Finally, a test data set can be used to provide an unbiased evaluation of a final model fit on the training data set.

To generate one or more treatment plans and/or implant designs, the third-party data can be input into the trained machine learning model(s). Additional data, such as the selected subset of the third-party data sets and/or similar patient data sets (e.g., electronic medical records, patient images, etc.), and/or treatment data from the selected subset, can also be input into the trained machine learning model(s). The trained machine learning model(s) can then calculate whether various candidate treatment procedures and/or medical device designs (e.g., patient-specific orthopedic implants) are likely to produce a favorable outcome for the patient, or meet one or more parameters (e.g., coverage parameters, reimbursement parameters, regulatory parameters, or the like). Based on these calculations, the trained machine learning model(s) can select data, data sources, etc. used to generate treatment plans. Additionally or alternatively, based on these calculations, the trained machine learning model(s) can select at least one treatment plan or implant design for the patient. In embodiments where multiple trained machine learning models are used, the models can be run sequentially or concurrently to compare outcomes and can be periodically updated using training data sets. The third-party collection module can use one or more of the machine learning models based the model's predicted accuracy score.

The third-party data collected and analyzed by the third-party collection module can be transmitted via the communication network 104 to the client computing device 102 for output to a user (e.g., clinician, surgeon, healthcare provider, patient). The client computing device 102 can notify a user that third-party data is available, found, collected, and/or analyzed. The third-party data can be used to create or modify a biomechanical virtual model of the patient, determine one or more motion parameters associated with the patient, and/or generate a surgical plan.

The client computing device 102 can periodically query data sources (e.g., remote servers, databases, websites, etc.) to determine whether new data is available. The newly available data can be analyzed to determine whether it may be clinically relevant to planned treatments. If so, the client computing device 102 can collect and analyze the newly available data to determine whether the planned treatment should be modified based on the new data. In response to determining that a user should review the data or the planned treatment should be modified, the client computing device 102 can send a notification to the user based on the newly available data. In some embodiments, the notification can indicate that newly available data could be used to modify a planned treatment. In some embodiments, the notification can include a modified treatment plan based on the newly available data. A user can compare the previous planned treatment to the modified planned treatment to determine which planned treatment to pursue. For example the display 122 can concurrently display the previous planned treatment, the modified planned treatment, and differences between the two. This process can be used to modify treatment plans, implant designs, or the like.

The display 122 can include a graphical user interface (GUI) for visually depicting various aspects of third-party data, including similarities or differences in comparison to data of the patient. The client computing device 102 can further include one or more user input devices (not shown) allowing the user to modify, select, approve, and/or reject the displayed treatment plan(s), data sources, sets of data, etc. The display 122 can display the source of the third-party data, selectors (e.g., selector boxes, menus, etc.) for selecting or deselecting the sources (e.g., selecting for detailed review of third-party data), or the like. The display 122 can then display third-party data only from user-selected sources. This allows a user to navigate available data sources that are trusted by the user. Selected third-party sources can be linked to the system for continuous or periodic queries. If the third-party data source is updated, the system can automatically search the third-party data source to determine whether newly available data should be used. A notification can be sent to the user if the third-party data source becomes unavailable.

In some embodiments, the user can select or deselect sets of data. If the user deselects a set of third-party data (e.g., a set of data based on a particular population group), a treatment plan can be updated without using the deselected third-party data. In some embodiments, the display 122 can show different treatments based on whether third-party data sets or sources are used. In some embodiments, the user can select or deselect types of data (e.g., images, text, reference patient data, data from studies, etc.) to be collected. In some embodiments, the user or system can select sets of metrics/parameters from treatment plans to generate a new treatment plan for achieving the selected metrics/parameters.

The display 122 can also include controls for receiving data from remote or third-party applications or programs. For example, a user can select one or more treatment planning applications on remote servers, and those selected applications can generate treatment plans, which can be consolidated into a single plan. In some embodiments, the display 122 can display a first plan for intervertebral cages manufactured by a first company. The display 122 can concurrently or sequentially display a second plan for instruments or surgical equipment from a second company. The display 122 can show a single surgical procedure plan with post-operative outcomes associated with the first and/or second plans.

To facilitate visualization, the display 122 can display one or more patient images, virtual models, and associated metrics/parameters, including pre-operative metrics/parameters, planned metrics/parameters, and/or post-operative metrics/parameters. As another example, the display 122 can show a design for a medical device to be implanted in the patient, such as a 2D or 3D model of the device design. The device design can include, for example, a size and configuration of an implant (e.g., footprint of intervertebral cage), length and curvature of spinal rods, length and characteristics of screws (e.g., bone screws, pedicle screws), range of motion of artificial discs, or the like. The display 122 can also show patient information, such as 2D or 3D images or models of the patient's anatomy where the surgical procedure is to be performed and/or where the device is to be implanted. In some procedures, the system can design implants based on received third-party data related to the size of the implant. For example, the size of the implant (e.g., an interbody cage or an artificial disc) can be a percentage of vertebral endplate(s), percentage of original disc height prior to surgery, or the like. In some procedures, the system can design implants based on received third-party data related to the curvature of the patient's spine. For example, a length, curvature, and strength of the implants (e.g., spinal rods) can be designed based on the received third-party curvature data.

The display 122 can display recommended types of implants, procedures (e.g., levels of treatment in spine procedures), placement criteria, contact interface criteria, loading criteria, properties of implants, characteristics of implants, or the like. In spine procedures, the type of implant can be, for example, a fixed intervertebral cage, an artificial disc, a rod, etc. selected based on whether the system determines there should be mobility at spinal levels. The placement criteria can include, for example, distance from anatomical features, position with respect to the perimeter of a vertebral endplate or body, and/or other positional location. In some spinal fusion procedures, the coverage criteria can include, for example, maximum or minimum percentage of coverage of the vertebral endplate, areas of coverage along the vertebral endplate, or the like. The load-bearing characteristics can include, for example, strength of the implant, fracture toughness of the implant, fatigue life of implant, degrees of motion of the implant, or the like. The characteristics of the implant can include, for example, lattice characteristics (e.g., density of lattice structure, distribution of lattice structure), openings for receiving grafting material, or the like. The display 122 can display recommendations for other procedures.

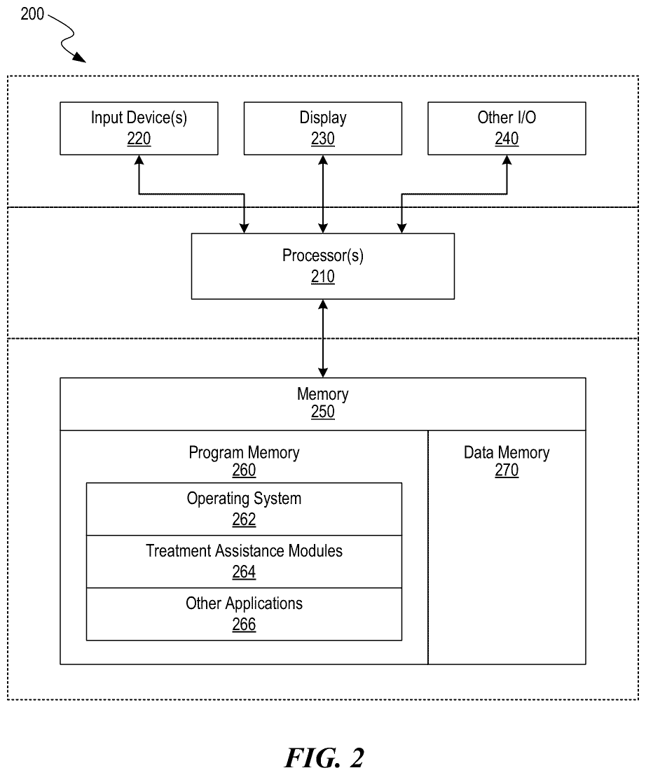

FIG. 2 illustrates a computing device 200 suitable for use in connection with the system 100 of FIG. 1, according to an embodiment. The computing device 200 can be incorporated in various components of the system 100 of FIG. 1, such as the client computing device 102 or the server 106. The computing device 200 includes one or more processors 210 (e.g., CPU(s), GPU(s), HPU(s), etc.). The processor(s) 210 can be a single processing unit or multiple processing units in a device or distributed across multiple devices. The processor(s) 210 can be coupled to other hardware devices, for example, with the use of a bus, such as a PCI bus or SCSI bus. The processor(s) 210 can be configured to execute one more computer-readable program instructions, such as program instructions to carry out of any of the methods described herein.

The computing device 200 can include one or more input devices 220 that provide input to the processor(s) 210, e.g., to notify it of actions from a user of the device 200. The actions can be mediated by a hardware controller that interprets the signals received from the input device and communicates the information to the processor(s) 210 using a communication protocol. Input device(s) 220 can include, for example, a mouse, a keyboard, a touchscreen, an infrared sensor, a touchpad, a wearable input device, a camera- or image-based input device, a microphone, or other user input devices.

The computing device 200 can include a display 230 used to display various types of output, such as text, models, virtual procedures, surgical plans, implants, graphics, and/or images (e.g., images with voxels indicating radiodensity units or Hounsfield units representing the density of the tissue at a location). In some embodiments, the display 230 provides graphical and textual visual feedback to a user. The processor(s) 210 can communicate with the display 230 via a hardware controller for devices. In some embodiments, the display 230 includes the input device(s) 220 as part of the display 230, such as when the input device(s) 220 include a touchscreen or is equipped with an eye direction monitoring system. In alternative embodiments, the display 230 is separate from the input device(s) 220. Examples of display devices include an LCD display screen, an LED display screen, a projected, holographic, or augmented reality display (e.g., a heads-up display device or a head-mounted device), and so on.

Optionally, other I/O devices 240 can also be coupled to the processor(s) 210, such as a network card, video card, audio card, USB, firewire or other external device, camera, printer, speakers, CD-ROM drive, DVD drive, disk drive, or Blu-Ray device. Other I/O devices 240 can also include input ports for information from directly connected medical equipment such as imaging apparatuses, including MRI machines, X-Ray machines, CT machines, etc. Other I/O devices 240 can further include input ports for receiving data from these types of machine from other sources, such as across a network or from previously captured data, for example, stored in a database.

In some embodiments, the computing device 200 also includes a communication device (not shown) capable of communicating wirelessly or wire-based with a network node. The communication device can communicate with another device or a server through a network using, for example, TCP/IP protocols. The computing device 200 can utilize the communication device to distribute operations across multiple network devices, including imaging equipment, manufacturing equipment, etc.

The computing device 200 can include memory 250, which can be in a single device or distributed across multiple devices. Memory 250 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory 250 is a non-transitory computer-readable storage medium that stores, for example, programs, software, data, or the like. In some embodiments, memory 250 can include program memory 260 that stores programs and software, such as an operating system 262, one or more treatment assistance modules 264, and other application programs 266. The treatment assistance module(s) 264 can include one or more modules configured to perform the various methods described herein (e.g., the data analysis module 117 and/or treatment planning module 118 described with respect to FIG. 1). Memory 250 can also include data memory 270 that can include, e.g., reference data, configuration data, settings, user options or preferences, etc., which can be provided to the program memory 260 or any other element of the computing device 200.

### B. Select Methods for Determining and Implementing Patient-Specific Diagnostic Plans

As set forth above, the present technology includes systems and methods for providing patient-specific medical care. In some embodiments, the systems and methods include determining a patient-specific diagnostic plan, performing/implementing the patient-specific diagnostic plan, and then, after performing the patient-specific diagnostic plan, designing a patient-specific treatment or surgical plan based at least in part on the outcome of the patient-specific diagnostic plan. In this way, the present technology is expected to improve patient health by ensuring that patient-specific treatment plans are designed based off of appropriate diagnostic information.

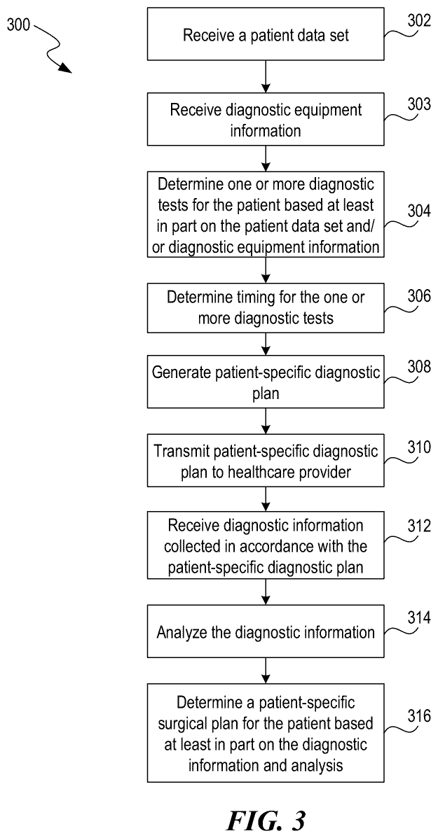

FIG. 3 is a flow diagram illustrating a method 300 for treating a patient including evaluating the patient's medical condition, such as by determining and/or implementing a patient-specific diagnostic plan. Some or all of the method 300 can be performed by various computing systems or software modules, including, for example, the computing systems and software modules described above with respect to FIGS. 1 and 2.

The method 300 can begin at block 302 by receiving a patient data set for a particular patient in need of medical treatment. The patient data set can be received at a server, computing device, or other computing system. For example, in some embodiments the patient data set can be received by the server 106 shown in FIG. 1. In some embodiments, the computing system that receives the patient data set at block 302 also stores one or more software modules (e.g., the diagnostic planning module 115, the modeling module 116, the data analysis module 117, the treatment planning module 118, the disease progression module 120, and/or the intervention timing module 121, shown in FIG. 1, or additional software modules for performing various operations of the method 500).

The patient data set can include data representative of the patient's demographics and health. The patient demographic information can include patient identification number (ID), name, age, sex, height, weight, body mass index (BMI), occupation, activity level, health rating, or the like. The health information can include data representative of the patient's condition, anatomy, pathology, symptoms, comorbidities, medical history, preferences, and/or any other information or parameters relevant to the patient. Such data can include previous surgical intervention data, previous treatment outcome data, progress data (e.g., surgeon notes), patient feedback (e.g., feedback acquired using quality of life questionnaires, surveys), clinical data, vital signs, medication information, allergies, or the like. In some embodiments, the patient data set can also include image data, such as camera images, Magnetic Resonance Imaging (MRI) images, ultrasound images, Computerized Aided Tomography (CAT) scan images, Positron Emission Tomography (PET) images, X-Ray images, and the like. Although the data set received at block 302 can include any of the above-identified data, in some embodiments the data set received at block 302 need only include general patient demographic information (e.g., age, sex, BMI, health rating) and general information about the patient's health (e.g., symptoms, pain location, pain intensity, etc.). As described below, this information can be used to determine a patient-specific diagnostic plan for the patient.

In some embodiments, the method 300 can also include receiving diagnostic equipment information at block 303. The diagnostic equipment information can be received at the same server, computing device, or other computing system that received the patient data set at block 302 (e.g., the server 106 shown in FIG. 1). The diagnostic equipment information can include information about the equipment available for use at the healthcare provider location (e.g., hospital, ambulatory surgical center (ASC), pain clinic, etc.) treating the patient. For example, the diagnostic equipment information can include, but is not limited to, available imaging systems (e.g., MRI, fMRI, X-Ray, CT, ultrasound, etc.), other diagnostic systems (e.g., EMG machine, NCV machine, etc.), diagnostic instruments (e.g., inclinometer, goniometer, etc.), etc. The diagnostic equipment information can also include specific information about the available systems and instruments, such as manufacturer, model number, specifications, user-selected settings/configurations, or the like.

The method 300 can continue at block 304 by determining one or more diagnostic tests for the patient based at least in part on the patient data set received at block 302 and/or the diagnostic equipment information received at block 303. The diagnostic tests determined at block 304 can include a specific set of diagnostic tests aimed to collect specific diagnostic information that is expected to be useful in determining a treatment plan for the patient. The operation of determining the one or more diagnostic tests can therefore include first determining the specific diagnostic information expected to be useful to determine a treatment plan for the patient, and then identifying one or more diagnostic tests for obtaining the diagnostic information. The diagnostic tests can include, but are not limited to, different types of imaging tests, pain tests/questionnaires, overall health tests/questionnaires, range of motion tests, gait and other motion tests, nerve tests, bone tests, strength tests, stability tests, cerebral spinal fluid tests, blood tests, sleep tests, and other miscellaneous tests. The diagnostic information can include the test results for any of the foregoing diagnostic tests, and/or physiological parameters that can be calculated from the test results for any of the foregoing diagnostic tests. Additional details of example diagnostic tests that may be identified in the operation at block 304 are described below with reference to FIGS. 4A and 4B.

As set forth above, determining the one or more diagnostic tests at block 304 can include determining specific diagnostic information that is expected to be needed to design an optimal treatment plan for the patient. The specific diagnostic information can be determined based on a patient's health and demographics, and therefore may be different depending on the individual patient's health and demographics. As a non-limiting example, a first patient who is a 40-year-old overweight male complaining of intense lower back pain may require different specific diagnostic information to determine an optimal treatment plan, and thus require a different set of diagnostic tests than, a second patient who is a 65-year-old female complaining of sporadic pain and numbness. The operation at block 304 can therefore identify the particular diagnostic tests that should be conducted on a particular patient to obtain the needed specific diagnostic information. In this way, determining the diagnostic tests at block 304 is a “patient-specific” operation. Without intending to be bound by theory, identifying the specific diagnostic information needed, and the specific diagnostic tests to be performed to obtain the needed specific diagnostic information, is expected to (1) preserve medical resources and lower costs by reducing the amount of unnecessary diagnostic tests performed on patients, and (2) improve patient outcomes by ensuring that the correct diagnostic tests are performed on the patient, and therefore appropriate diagnostic information is obtained, before determining a treatment plan.

In some embodiments, the patient data set received at block 302 may include some diagnostic information in addition to demographic and health data. For example, as described above, the patient data set received at block 302 may include patient image data, measurements, or the like, such as from an initial clinical evaluation of the patient. In such embodiments, the operation in block 304 can account for the diagnostic information already obtained. For example, if the patient data received at block 302 includes X-ray image data, the operation at block 304 would account for this and would generally not recommend obtaining the same X-ray images to avoid unnecessarily duplicative diagnostic tests. Instead, the operation at block 304 would analyze the data, including the diagnostic information received with the patient data set, to determine what additional diagnostic information (if any) is necessary to determine the optimal treatment plan for the patient.

In some embodiments, the operation at block 304 includes prescribing diagnostic tests that are capable of being performed at the healthcare provider location treating the patient. This can include analyzing the diagnostic equipment information received at block 303 either before, during, or after identifying the one or more diagnostic tests. If a healthcare provider location is unable to perform a recommended one of the diagnostic tests based on the diagnostic equipment information, a different diagnostic test can be recommended.

In some embodiments, the operation at block 304 can also account for user-specific preferences. For example, if a healthcare provider requests that a surgical plan for a patient include disease progression simulations, the operation at block 304 can incorporate this request by recommending one or more diagnostic tests that may be useful for modeling disease progression predictions.

In some embodiments, the operation at block 304 can be performed by the diagnostic planning module 115 of the system 100 (FIG. 1). That is, the diagnostic planning module 115 can automatically analyze the patient data and/or diagnostic equipment information to identify the one or more diagnostic tests. In some embodiments, the diagnostic planning module 115 can include one or more trained machine learning models or artificial intelligence architectures that can assist in identifying the one or more diagnostic tests. In such embodiments, the patient data and diagnostic equipment can represent the “inputs” to the machine learning model, and the diagnostic information and/or the one or more diagnostic tests can represent the “outputs” of the machine learning model. The model can be trained using reference patient data sets, as described previously.

Once the one or more diagnostic tests are determined at block 304, the method 300 can continue at block 306 by determining a timing for the diagnostic tests. Determining the timing for the one or more diagnostic tests can include identifying a particular date range within which the diagnostic test should be performed (e.g., within 1 month, within 2 months, within 3 months, within 6 months, etc.). Determining the timing of the one or more diagnostic tests can also include determining a time of day the diagnostic test should be performed (e.g., in the morning, in the evening, at night, etc.), in addition to or in lieu of determining the date range the diagnostic tests should be performed within. In some embodiments, the operation at block 306 can be performed by the diagnostic planning module 115 of the system 100 (FIG. 1). For example, in embodiments in which the diagnostic planning module 115 includes a machine learning module for outputting the diagnostic information and/or diagnostic tests, the same or different machine learning model may also output a timing for the diagnostic tests.

The method 300 can continue at block 308 by generating a patient-specific diagnostic plan (also referred to herein simply as the “diagnostic plan”). The diagnostic plan can include the one or more diagnostic tests identified at block 304, the timing for the one or more diagnostic tests identified at block 306, and/or instructions for performing the one or more diagnostic tests. The diagnostic plan can be displayed as a digital report, printed as a hard copy report, or otherwise generated for user review. In some embodiments, generating the diagnostic plan can include generating computer-readable instructions that, when executed by a processor, generate a digital version of the diagnostic plan for user review. In some embodiments, the operation at block 308 can be performed by the diagnostic planning module 115 of the system 100 (FIG. 1).

After the patient-specific diagnostic plan has been generated, the method 300 can continue at block 310 by transmitting the patient-specific diagnostic plan to a healthcare provider caring for the patient. In some embodiments, the same computing system used at blocks 302-308 to generate the diagnostic plan can transmit the diagnostic plan to a computing device for surgeon review (e.g., the client computing device 102 described in FIG. 1). This can include directly transmitting the diagnostic plan to the computing device or uploading the diagnostic plan to a cloud or other storage system for subsequent downloading. In some embodiments, the diagnostic plan or features included within the diagnostic plan can be transmitted to multiple user devices to coordinate at-home diagnostic testing, primary physician testing, specialist testing, or other testing.

The healthcare provider can then review and execute the patient-specific diagnostic plan. This may include, for example, performing the one or more diagnostic tests prescribed in the diagnostic plan to obtain the specific diagnostic information. Once the diagnostic tests have been performed, the healthcare provider can upload the diagnostic information (e.g., the test results) to a server or otherwise transmit the results back to the computing system performing the method 300. Thus, the method 300 can continue at block 312 by receiving the diagnostic information collected in accordance with the patient-specific diagnostic plan. This may include, for example, receiving results from the diagnostic tests that were performed on the patient.

After receiving the diagnostic information, the method 300 can continue by analyzing the diagnostic information at block 314. This may include extrapolating additional diagnostic information from the received diagnostic information. For example, in some embodiments the diagnostic information received at block 312 may include image data of the patient's spine under various conditions (e.g., load-bearing vs. non-load bearing, flexion vs. neutral vs. extension, etc.). At block 314, the method 300 can include analyzing the image data to determine additional diagnostic information such as various physiologic metrics under the various conditions. The physiologic metrics can include, but are not limited to, lumbar lordosis, Cobb angles, disc height, segment flexibility, coronal parameters (e.g., coronal balance, global coronal balance, coronal pelvic tilt, etc.), sagittal parameters (e.g., sacral slope, thoracic kyphosis, etc.) and/or pelvic parameters (e.g., pelvic incidence). The physiologic metrics can include micro-measurements (e.g., metrics associated with specific or individual segments of the patient's spine) and/or macro-measurements (e.g., metrics associated with multiple segments of the patient's spine). In some embodiments, the physiologic metrics can be calculated using one or more software modules of the computing system performing the method 300. In some embodiments, the physiologic metrics can be calculated in substantially real-time. That way, if the physiologic metrics cannot be determined from the provided diagnostic information (e.g., if pelvic incidence cannot be measured by the provided X-rays), then feedback (e.g., instructions for performing additional diagnostic tests such as obtaining additional X-rays from different views, instructions for retrieving data from a patient's medical record, digital filing cabinet, or other data source, etc.) can be sent to the healthcare provider in substantially real time. Diagnostic plans can therefore be updated in real-time or near real-time based on received diagnostic information. In some embodiments, updated diagnostic plans can be analyzed to determine whether additional diagnostic tests should be performed.

In some embodiments, the diagnostic information received at block 312 may already include one or more physiologic metrics. In such embodiments, it may not be necessary to calculate the physiologic metrics from the source data (e.g., the image data) at block 314. However, the physiologic metrics can nevertheless be calculated at block 314 to validate the physiologic metrics received at block 312. If the calculated physiologic metrics do not match the received physiologic metrics (or deviate by more than a predetermined threshold, such as deviate by more than 1%, 2%, 3%, 5%, or 10%), the discrepancy can be flagged for user review.

In some embodiments, the operation at block 314 includes generating a diagnostic-driven anatomical model based on the received diagnostic information and the associated analysis. This may include, for example, converting captured motion and/or image data into a two- or three-dimensional simulated virtual model of patient anatomy, as described above in detail with reference to FIG. 1.

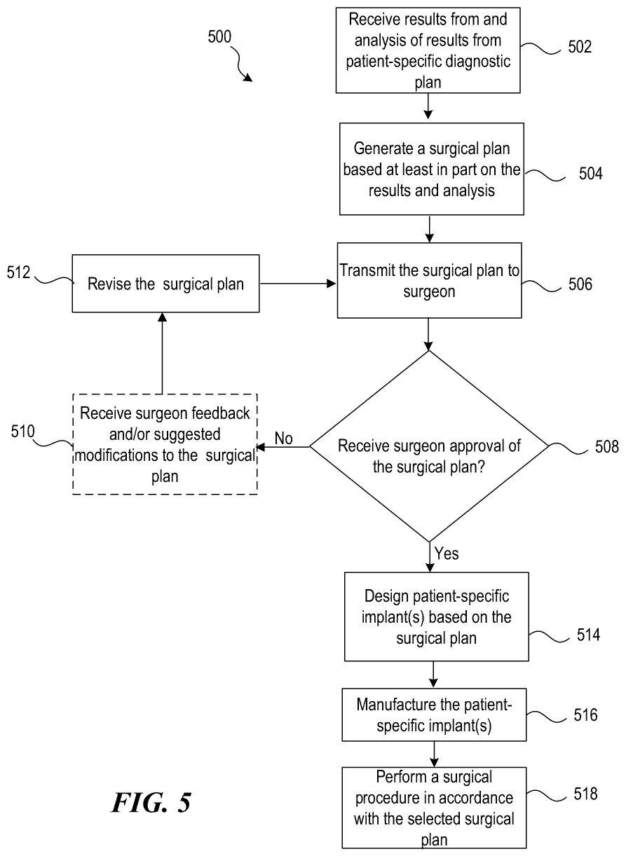

The method 300 can continue at block 316 by determining a patient-specific surgical plan for the patient based at least in part on the diagnostic information and associated analysis. That is, the diagnostic information obtained by performing the one or more diagnostic tests prescribed by the diagnostic plan can be used to inform a treatment protocol for the patient. In some embodiments, the operation at block 316 can be performed by the system 100 of FIG. 1, e.g., via the data analysis module 117 and the treatment planning module 118. Additional details regarding determining a patient-specific treatment plan for a patient based at least in part on the results of a patient-specific diagnostic plan are described below with reference to FIG. 5.

As one skilled in the art will appreciate from the disclosure herein, the method 300 can be implemented and performed in various ways. In some embodiments, the operations may be performed by a computing system such as the server 106 (FIG. 1), or another suitable computing system. Moreover, any of the foregoing blocks may also be implemented as computer-readable instructions stored in memory and executable by one or more processors of the associated computing system(s).

Further, in some embodiments various operations of the method 400 (e.g., the operations at blocks 304, 306, 308, 314, and/or 316) utilize one or more (e.g., trained) machine learning models/algorithms. For example, the machine learning models can determine diagnostic information needed to generate a surgical plan, determine diagnostic information needed to generate one or more anatomical models or predicted post-operative outcomes, determine suitable tests for determining the diagnostic information, or the like. The output from the machine learning model can therefore include a list of diagnostic information needed, one or more specific tests for collecting the diagnostic information, a timeframe for receiving the diagnostic information, and instructions for performing the one or more specific tests, or the like.

FIGS. 4A and 4B are tables that list examples of diagnostic tests that may be prescribed as part of the patient-specific diagnostic plans described herein. Referring to FIGS. 4A and 4B collectively, the diagnostic tests may include imaging tests 402, pain tests 404, overall health tests 406, range of motion tests 408, gait/motion tests 410, nerve tests 412, bone tests 414, strength tests 416, stability tests 418, cerebral-spinal fluid (CSF) tests 420, blood tests 422, and/or sleep tests 424.

The imaging tests 402 can include, but are not limited to, Magnetic Resonance Imaging (MRI), functional Magnetic Resonance Imaging (fMRI), Computed Tomography (CT), CT myelogram, Positron Emission Tomography (PET), discogram, x-ray, ultrasound, or other imaging modalities. The imaging tests 402 prescribed under the patient-specific diagnostic plans described herein may include one or more specific anatomical locations, one or more specific views, one or more specific patient positions, and/or one or more specific conditions. Example locations include cervical vertebrae, thoracic vertebrae, lumbar vertebrae, the sacrum, the pelvis, and soft tissue proximate any of the foregoing bony structures. Example views include anterior, posterior, lateral, superior, inferior, or combinations thereof. Example positions include supine, prone, standing, under flexion, under extension, neutral, side-bending, etc. Example conditions include load-bearing, non-load bearing, etc. As a particular example, the patient-specific diagnostic plans may prescribe, and include instructions for obtaining, anterior, posterior, and lateral X-ray images of the L1-L5 vertebrae while the patient is standing. As another particular example, the patient-specific diagnostic plans may prescribe, and include instructions for obtaining, MRI images of patient soft tissue proximate the C1-C4 vertebrae while the patient is in a neutral position, under flexion, and under extension. The foregoing examples are provided by way of example only—one skilled in the art will appreciate that the diagnostic plans herein can prescribe any combination of the foregoing locations, views, positions, and conditions.

The pain tests 404 include tests and questionnaires designed to assess a patient's pain. Example pain tests 404 include, but are not limited to, subjective pain tests using, e.g., the Numerical Rating Scale (NRS), the Visual Analog Scale (VAS), the Acute Low Back Pain Screening Questionnaire (ALBPSQ), the Defense and Veterans Pain Rating Scale (DVPRS), the Adult Non-Verbal Pain Scale (NVPS), the Critical Care Observation Tool (CPOT), the Behavioral Pain Scale (BPS), the Pain Assessment in Advanced Dementia Scale (PAINAD), or other suitable pain scales. In embodiments in which a pain test is prescribed under the patient-specific diagnostic plan, the plan can recommend a specific test/scale to use. For example, a first patient-specific diagnostic plan for a first patient may prescribe using the NRS scale, while a second patient-specific diagnostic plan for a second patient may prescribe using the VAS scale.

The overall health tests 406 include tests and questionnaires designed to assess patient quality of life and overall health, which may or may not include patient pain levels. Example overall health tests 406 include, but are not limited to, the Oswestry Disability Index (ODI), the Quebec Back Pain Disability Scale (QBPDS), the Fear-Avoidance Beliefs Questionnaire (FABQ), the STarT Back Screening Tool, the Roland-Morriss Disability Questionnaire (RMDQ), or a Quality of Life (QOL) questionnaire. In embodiments in which a health test is prescribed under the patient-specific diagnostic plan, the plan can recommend a specific test/scale to use. For example, a first patient-specific diagnostic plan for a first patient may prescribe using the QBPDS test, while a second patient-specific diagnostic plan for a second patient may prescribe using the RMDQ.

The range of motion tests 408 include tests designed to identify patient hypomobility or hypermobility. Example range of motion tests 408 include, but are not limited to, measuring active range of motion (AROM) or passive range of motion (PROM), and can include measuring range of motion in various planes including flexion, extension, side-bending, and rotation. The range of motion tests can also include specific tests useful in determining range of motion, such as the “fingertip to floor” test, the Schober test, the modified Schober test, or the like. In some embodiments, the range of motion tests 408 can be performed using radiography or other imaging. In other embodiments, the range of motion tests 408 can be performed manually with the use of a diagnostic instrument (e.g., an inclinometer, goniometer, tape measure, etc.). Similar to the imaging tests 402, the range of motion tests 408 prescribed under the patient-specific diagnostic plans described herein may include one or more specific anatomical locations, one or more specific patient positions, and/or one or more specific conditions. Specific anatomical locations can include vertebral ranges within or spanning the cervical vertebrae, thoracic vertebrae, and/or lumbar vertebrae. Specific positions can include standing, sitting, prone, supine, etc. Specific conditions can include load-bearing vs. non-load bearing. Thus, as a non-limiting example, the patient-specific diagnostic plans may prescribe, and include instructions for obtaining, measuring range of motion for flexion, extension, side-bending, and rotation for the lumber region.

The gait/motion tests 410 include tests designed to identify issues with a patient's ambulation or other movement. The gait/motion tests 410 can include, but are not limited to, gait analysis, movement control tests, functional gait assessment, dynamic gait index, six-minute walk test, timed up-and-go test, step test, two chair test, sit-stand test, or the like. Gait analysis can include measuring gait parameters, such as gait speed, gait cycle, step time, stride length, or the like.

The nerve tests 412 include tests designed to examine the health of a patient's nerve or nervous system. The nerve tests 412 can include electrical-based tests including electromyography (EMG), nerve conduction velocity (NCV), or the like. The nerve tests 412 can also include non-electrical tests, such as the Straight Leg Raise (SLR) test, the Crossed Straight Leg Raise (CSLR) test, the Femoral Nerve Tension Test, the Slump Test, or the like. The nerve tests 412 can also include tests for detecting loss of sensation or numbness, such as the pin-prick test. The nerve tests 412 prescribed under the patient-specific diagnostic plans described herein may also include one or more specific anatomical locations or structures (e.g., a specific nerve or nerve bundle) for the test to be performed.

The bone tests 414 include tests designed to examine the health of patient's bones. Example bone tests include, but are not limited to, bone density tests such as a dual-energy X-ray absorptiometry (DEXA) or CT-based bone density test, which can be used to identify a patient's T-score and Z-score to identify if the patient has osteoporosis or another condition that impacts bone density. The bone tests 414 can also include a bone scan (i.e., a bone scintigraphy) to image bone metabolism. The bone tests 414 can also include biopsies (e.g., bone and/or bone marrow biopsies). Similar to the other tests described herein, the bone tests 414 prescribed under the patient-specific diagnostic plans described herein may include a specific anatomical location or structure (e.g., a specific bone) for the test to be performed.

The strength tests 416 include tests designed to measure muscle strength and/or muscle loss. Example strength tests include, but are not limited to, the 90-90 Straight Leg Raise Test, the Ober Test, the Rectus Femoris Test, and the Thomas Test. Similar to the other tests described herein, the strength tests 416 prescribed under the patient-specific diagnostic plans described herein may include a specific anatomical location or structure (e.g., one or more specific muscles) for the test to be performed.

The stability tests 418 include tests designed to identify instability within the patient, such as instability within the patient's spine. Example stability tests include, but are not limited to, the H&I Test, the Passive Lumbar Extension Test, the Prone Segmental Instability Test, and the Specific Lumbar Torsion Test. The stability test 418 prescribed under the patient-specific diagnostic plans described herein may include a specific anatomical location or structure (e.g., one or more specific vertebrae or vertebral segments) for the test to be examine.

The CSF tests 420 are used to analyze a patient's cerebral spinal fluid to detect certain conditions and disease. Example procedures for collecting a CSF sample include a spinal tap (lumbar puncture), a cisternal puncture, and a ventricular puncture. Example metrics that can be analyzed in a CSF sample include, but are not limited to, opening pressure, appearance, cell count, concentration of various substances (e.g., chloride, protein, glucose, glutamine, lactate dehydrogenase, etc.), and ratios between the various substances and CSF (e.g., glucose:CSF serum ratio).

The blood tests 422 are used to analyze a patient's blood to detect certain conditions and diseases. Example blood tests 422 include, but are not limited to, complete blood counts (CBC), basic metabolic panel, comprehensive metabolic panel, lipid panel, thyroid panel, coagulation panel, cardiac biomarkers, liver function tests (LFTs), kidney function tests (KFTs), and the like.

The sleep tests 424 are used to identify patient sleep quality and issues associated with patient sleep. Example sleep test include, but are not limited to, polysomnography, home respiratory polygraphy, actigraphy, and sleep questionnaires.

As one skilled in the art will appreciate, the various diagnostic tests shown in FIGS. 4A and 4B and described above are not intended to be exhaustive. The patient-specific diagnostic plans can include variations of any of the diagnostic tests described herein, and/or can include other diagnostic tests not expressly described herein. As described throughout this Detailed Description, the results of the tests listed in FIGS. 4A and 4B are expected to provide diagnostic information that may be useful in determining patient-specific surgical plans.

### C. Select Methods for Generating and Implementing Patient-Specific Treatment Plans Based on Patient-Specific Diagnostic Plans

FIG. 5 is a flow diagram illustrating a method 500 for providing patient-specific medical care using the patient-specific diagnostic plans described herein, according to an embodiment of the present technology. Some or all of the method 500 can be performed by various computing systems or software modules, including, for example, the computing systems described above with respect to FIGS. 1 and 2.

The method 500 can begin at block 502 by receiving the results from the patient-specific diagnostic plan and/or any analysis of the results from the patient-specific diagnostic plan. The results from the patient-specific diagnostic plan can include diagnostic information collected via the diagnostic tests prescribed via the patient-specific diagnostic plan, as described above with reference to block 312 of the method 300 (FIG. 3). The analysis of the results can include any subsequent analysis performed on the results, as described above with reference to block 314 of the method 300 (FIG. 3). In some embodiments, the results and/or associated analysis can be received at a server, computing device, or other computing system. For example, in some embodiments the results and/or associated analysis can be received by the server 106 shown in FIG. 1. In some embodiments, the computing system that receives the results at block 502 also stores one or more software modules (e.g., the diagnostic planning module 115, the diagnostic-driven anatomical modeling module 116, the data analysis module 117, the treatment planning module 118, the disease progression module 120, and/or the intervention timing module 121, shown in FIG. 1, or additional software modules for performing various operations of the method 500). In some embodiments, the analysis can be stored performed by the computing system, such that the analysis is not “transmitted” to the computing system (e.g., from a healthcare provider). Accordingly, although described as “receiving,” one skilled in the art will appreciate that the operation at block 502 can include accessing the results and analysis that may have been previously uploaded or saved to a local database.

The method 500 can continue at block 504 by generating a surgical plan based at least in part on the results of the patient-specific diagnostic plan and/or the analysis of the results of the patient-specific diagnostic plan received at block 502. In some embodiments, the surgical plan can also be based at least in part on other patient data, such as patient demographic and/or health data included within the patient data set described above with reference to block 302 of the method 300 (FIG. 3). In some embodiments, the surgical plan can include diagnostic information obtained using the patient-specific diagnostic plan. The diagnostic information can include, for example, scores (e.g., stretch scores, sit and twist scores, strength scores, motion scores, spinal stability scores, pre-operative pain scores, physical examination scores, etc.), diagnostic test data, or other data disclosed herein. The surgical plan can identify correlations between the diagnostic data, simulation data, and/or features of the surgical plan. For example, the surgical plan can identify a level of the spine for a fusion procedure based on that level having a spinal-level stability score below a threshold level, contributing a threshold amount to a low overall-spine stability score, and/or user selection.

As described in detail below, the surgical plan can include a target location or region of interest for surgical intervention and one or more surgical procedures or interventions to be performed at the region of interest. The surgical plan can also include predicted post-operative data associated with performing the surgical procedure at the target location. For example, the surgical plan may include a predicted or target post-operative anatomical configuration shown as a two- or three-dimensional virtual model. In some embodiments, the surgical plan also includes additional predicted post-operative analytics, such as predicted disease progression, predicted patient satisfaction, predicted patient mobility, predicted patient pain, predicted patient quality of life, etc.

In some embodiments, the operation of generating the surgical plan includes identifying a specific target location to be involved in the surgical procedure. For example, in the context of spinal surgery, generating the surgical plan may include identifying one or more vertebral levels for surgical intervention. In some embodiments, the vertebral level is a cervical vertebral level (e.g., C1-C5), a thoracic vertebral level (e.g., T1-T12), a lumbar vertebral level (e.g., L1-L5), and/or the sacrum. In some embodiments, the identified target location includes a specific range of vertebral levels to be involved in a surgery (e.g., L1-L3, L3-L5, L4-T12, C1-C3, etc.). The identified target location may include two, three, four, five, or more vertebral levels. Of course, the foregoing target locations are provided by way of example only, and the present technology is not limited to the anatomical locations listed above. Indeed, in some embodiments the target location may include anatomical structures other than the spine, such as the hip, knee, ankle, shoulder, elbow, wrist, hand, the jaw, the skull, or other anatomical locations, as described throughout this Detailed Description.

The target location can be identified by reviewing the diagnostic information and/or image data of the patient. In some embodiments, a computing system (e.g., the server 106 of FIG. 1) and/or one or more software modules (e.g., the treatment planning module 118 of FIG. 1) can review and analyze patient diagnostic information image data and automatically identify the target location. In such embodiments, a trained machine learning program or other software-based program can analyze patient image data, extract measurements from the patient image data, compare the extracted measurements to reference data (e.g., predetermined thresholds or ranges associated with “healthy” patients normalized for age, sex, gender, etc.), and identify anatomical regions that are candidates for surgical correction. Alternatively or additionally, the target location can be identified and/or confirmed through other suitable means, such as via a technician or healthcare provider reviewing image data and identifying anatomical deformities.

As provided above, in some embodiments the operation of generating the surgical plan also includes identifying a surgical procedure for the patient. In embodiments in which the surgical plan includes identifying a target location, the surgical procedure can be associated with the target location. In the context of spinal surgery, representative surgical procedures include spinal fusion, artificial disc replacement, vertebroplasty, kyphoplasty, spinal laminectomy/decompression, discectomy, facetectomy, foraminotomy, or other spine surgery procedures. Examples of spinal fusion surgery include posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). The foregoing are provided by way of example only, and the present technology can include identifying any type of spinal or other surgical procedures at block 504.

The surgical procedure associated with the surgical plan can be identified using any of the methods and systems described herein. For example, in some embodiments the server 106 of FIG. 1 and/or the associated software modules (e.g., the treatment planning module 118) can identify one or more surgical procedures based on, for example, user input, the received or extracted patient data, and/or the identified target location(s). For example, if the server 106 determines that a patient is suffering from disc degeneration from L3-L5, the server 106 may recommend a PLIF procedure to fuse L2-T12. Alternatively, the server 106 may recommend an artificial disc replacement at L3-L4 and L4-L5 to correct the degeneration while preserving motion. The surgical procedure can be identified using other methods and systems as well.

In some embodiments, the operation at block 504 can include reviewing and/or analyzing multiple types of surgical procedures and/or surgical steps to identify the surgical procedure for inclusion within the surgical plan. Types of surgical procedures and/or surgical steps can be selected for inclusion with the surgical plan (or eliminated from inclusion with the surgical plan) based on, for example, user input, insurance coverage of the procedure or step, healthcare provider parameters (e.g., based on healthcare provider ranking/scores such as hospital/physician expertise, number of similar procedures performed, hospital ranking for procedure, etc.), healthcare resource parameters (e.g., diagnostic equipment, facilities, surgical equipment such as surgical robots), and/or other non-patient related information (e.g., information that can be used to score, predict outcomes and risk profiles for procedures for the present healthcare provider, and/or rank procedures).

In some embodiments, the operation of generating the surgical plan includes identifying or designing a corrected anatomical configuration for the patient (the corrected anatomical configuration can also be referred to herein as the “planned configuration,” “optimized geometry,” “post-operative anatomical configuration,” or “target outcome”). The corrected anatomical configuration can reflect the desired and/or predicted anatomy of the patient if the surgical plan were performed. In some embodiments, generating the surgical plan includes generating one or more virtual models (two-dimensional models, three-dimensional models, etc.) showing the corrected anatomical configuration. The virtual model may include some or all of the patient's anatomy within the target location (e.g., any combination of tissue types including, but not limited to, bony structures, cartilage, soft tissue, vascular tissue, nervous tissue, etc.). In some embodiments, the corrected anatomical configuration is identified/determined before the surgical procedure and/or target location. That is, a computing system or user can model a preferred anatomical outcome, and, based on the desired anatomical outcome, identify a surgical procedure and target location that will achieve the desired anatomical outcome once performed.

In embodiments in which the operation of generating the surgical plan includes generating a virtual model, the virtual model can be based on the diagnostic information obtained from the patient-specific diagnostic plan. One or more surgical procedures can be simulated on the virtual model to generate simulated procedure data. The method 500 can include comparing the simulated procedure data with reference diagnostic data to determine a surgical procedure for the patient for inclusion with the surgical plan. As set forth above, machine learning algorithms can be used to perform the comparison. The reference diagnostic data can be prior patient data of patients that are similar to the current patient. A healthcare provider, machine learning system, and/or user can select the prior patients that are similar to the current patient, as previously described. The patient can also be tested periodically after surgery to collect post-operative diagnostic data, which can be used to retrain the ML algorithms to select more similar prior patients.

In some embodiments, generating the surgical plan includes generating one or more patient metrics associated with the corrected anatomical configuration. In the context of spinal surgery, patient metrics may include, for example, coronal parameters, sagittal parameters, pelvic parameters, Cobb angles, shoulder tilt, iliolumbar angles, coronal balance, lordosis angles, intervertebral space height, or other similar spinal parameters. Similar as described above, the patient metrics can be determined before identifying a surgical procedure and/or target location for surgical intervention. That is, a computing system or user can use the patient metrics to identify a surgical procedure and target location that will achieve the patient metrics once performed.

The surgical plan can include additional features. In some embodiments, for example, the surgical plan can include predicted disease progression, predicted patient satisfaction, predicted patient mobility, predicted patient pain, predicted patient quality of life, or the like. For example, the surgical plan may include estimates of disease progression if the patient were to undergo the identified surgical procedure at the identified target location. That is, the surgical plan can include virtual models (e.g., two-dimensional or three-dimensional virtual models) of patient anatomy at various intervals post-operation. For example, the surgical plan may include a predictive model of patient anatomy and other predictive data at one or more of 6 months post-op, 1 year post-op, 2 years post-op, 3 years post-op, 4 year post-op, 5 years post-op, 6 years post-op, 7 years post-op, 8 years post-op, 9 years post-op, and/or 10 years post-op. The disease progression model may also include predicted patient metrics (e.g., any of the patient metrics described herein, including coronal parameters, sagittal parameters, pelvic parameters, Cobb angles, shoulder tilt, iliolumbar angles, coronal balance, lordosis angles, intervertebral space height, or other similar spinal parameters) at any of the various post-operative intervals identified above, in addition to or in lieu of including the virtual model of predicted patient anatomy.

In some embodiments, the surgical plan can be based at least in part on one or more diagnostic-driven virtual models representing biomechanics of the patient. The diagnostic-driven virtual model can include model parameters (e.g., parametric parameters, relationships between model features, boundary conditions, etc.), model features (e.g., model surfaces, volumes, meshes for finite-element analysis, boundary conditions, constraints, kinematic analysis features, etc.), material properties, and other features determined using results from the diagnostic plan. For example, the model can include movable devices (e.g., implants, instruments, etc.), anatomic spinal elements, etc. The anatomical elements can include bony structures (e.g., vertebrae, vertebral bodies, etc.), soft tissue (e.g., ligaments), and other anatomical features that can be moved via drag-and-drop, modification of parametric parameters, etc. The virtual models can simulate, for example, a predicted post-operative range of motion based on the patient's pre-operative range of motion tests (e.g., range of motion tests 408 of FIG. 4A), gait/motion tests, etc. The diagnostic-driven virtual can be automatically updated based on newly received diagnostic data, thereby synchronizing virtual models and diagnostic plans. Diagnostic data can be ranked and utilized based on its ranking, thereby determining the relative contribution of diagnostic data to diagnostic-driven virtual models.

Once generated, the surgical plan can be digitally displayed as a surgical report on one or more display screens for ease of review, editing, annotation, the like. In some embodiments, the surgical plan can be stored as computer-executable instructions that can be executed via the surgical plan review module 123 on the client computing device 102 of FIG. 1. More specifically, the surgical plan can be reviewed using the surgical plan review program 125, shown in FIG. 1 and described in greater detail below with reference to FIGS. 5A-12.

In some embodiments, the operation of generating the surgical plan at block 504 includes generating a plurality of candidate surgical plans (or subsets of surgical plans such as surgical procedures), and then selecting the surgical from within the plurality of candidate surgical plans. For example, in some embodiments a computing system can automatically identify a plurality (e.g., two, three, four, five, six, seven, eight, nine, ten, or more) of surgical plans (or subsets of surgical plans such as surgical procedures) based on the patient-data set and/or one or more user-inputted criteria. The identified candidate surgical plans may be ranked and/or scored based on various factors, including predicted patient outcomes, user-review, etc. The highest ranked identified candidate surgical plans (e.g., based on predicted patient outcomes) can be selected as the surgical plan. In some embodiments, certain ranked surgical plans may not be selected as the surgical plan based on user review and/or failure to meet various user criteria. For example, if a particular surgical plan is identified as requiring a surgical procedure that the physician is unfamiliar with, the particular surgical plan may not be selected, and the method can instead include selecting the next best surgical plan as the surgical plan. Accordingly, in some implementations, physician-specific scoring is used to score candidate procedures/surgical plans before selecting the surgical plan. For example, procedures with scores meeting a threshold score (e.g., threshold post-operative metrics score, physician inputted threshold score, threshold outcome score, etc.) can be identified for user review. The system can therefore compare advantages and disadvantages of candidate procedures with respect to each other before selecting the surgical plan.

Once the surgical plan is generated at block 504, the method 500 can continue at block 506 by transmitting the surgical plan to a surgeon. In some embodiments, the same computing system used at blocks 502 and 504 can transmit the surgical plan to a computing device for surgeon review (e.g., the client computing device 102 described in FIG. 1). This can include directly transmitting the surgical plan to the computing device or uploading the first and second surgical plans to a cloud or other storage system for subsequent downloading.

The surgeon can review the surgical plan and, at block 508, approve or disapprove of the surgical plan. For example, the surgeon may review the surgical plan using the surgical plan reviewing program 125 (FIG. 1) to determine whether the surgeon deems the surgical plan acceptable. This may include, for example, reviewing the surgical plans target locations, surgical procedure, target/predicted post-operative anatomical configuration, and predicted post-operative patient metrics.

In some embodiments, the surgeon may not approve the surgical plan at block 508. In such embodiments, the surgeon can optionally provide feedback and/or suggested modifications to the surgical plan (e.g., by adjusting the virtual model or changing one or more aspects about the plan, providing comments on or more requested changes to the surgical plan, etc.). Accordingly, the method 500 can optionally include receiving (e.g., via the computing system) the surgeon feedback and/or suggested modifications at block 510. This may include, for example, modifying target locations for surgical intervention, surgical procedures, and/or target post-operative anatomical configuration. If surgeon feedback and/or suggested modifications are received at block 510, the method 500 can continue at block 512 by revising (e.g., automatically revising via the computing system) the surgical plan based at least in part on the surgeon feedback and/or suggested modifications received at block 510. In some embodiments, the method 500 can include requesting and obtaining additional diagnostic data for evaluating the surgeon feedback and/or suggested modifications. In some embodiments, the surgeon does not provide feedback and/or suggested modifications if they reject the surgical plan. In such embodiments, block 510 can be omitted, and the method 500 can continue at block 512 by revising (e.g., automatically revising via the computing system) the surgical plans by selecting new and/or additional reference patient data sets and/or generating a new candidate surgical plan. The revised and/or new surgical plan can then be transmitted to the surgeon for review. The operations at blocks 506, 508, 510, and 512 can be repeated as many times as necessary until the surgeon selects and approves a particular surgical plan.

Once surgeon approval of a surgical plan is received at block 508, the method 500 can continue at block 514 by designing (e.g., via the same computing system that performed blocks 502-508) one or more patient-specific implants based on the selected surgical plan. For example, the patient-specific implant can be designed based on the target location and surgical procedure included in the selected surgical plan. The patient-specific implant(s) can also be specifically designed such that, when implanted in the particular patient at the target location using the identified surgical procedure, it directs the patient's anatomy to occupy the target post-operative anatomical configuration (e.g., transforming the patient's anatomy from the patient's native anatomical configuration to the corrected anatomical configuration). The patient-specific implant can be designed such that, when implanted, it causes the patient's anatomy to occupy the corrected anatomical configuration for the expected service life of the implant (e.g., 5 years or more, 10 years or more, 20 years or more, 50 years or more, etc.). In some embodiments, the patient-specific implant is designed solely based on the virtual model of the corrected anatomical configuration and/or without reference to pre-operative patient images.

The patient-specific implant can be any of the implants described herein or in any patent references incorporated by reference herein. For example, the patient-specific implant can include one or more of screws (e.g., bone screws, spinal screws, pedicle screws, facet screws), interbody implant devices (e.g., intervertebral implants), cages, plates, rods, discs, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like. A patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.). Examples of patient-specific implants that can be designed at block 314 are described in U.S. application Ser. Nos. 16/048,167, 16/242,877, 16/207,116, 16/352,699, 16/383,215, 16/569,494, 16/699,447, 16/735,222, 16/987,113, 16/990,810, 17/085,564, 17/100,396, 17/342,329, 17/518,524, 17/531,417, 17/835,777, 17/851,487, 17/867,621, and 17/842,242, each of which is incorporated by reference herein in its entirety.

In some embodiments, designing the implant at block 516 can optionally include generating fabrication instructions for manufacturing the implant. For example, the computing system may generate computer-executable fabrication instructions that that, when executed by a manufacturing system, cause the manufacturing system to manufacture the implant.

In some embodiments, the patient-specific implant is designed at block 516 only after the surgeon has selected a surgical plan. Accordingly, in some embodiments, the implant design is neither transmitted to the surgeon with the surgical plan at block 508, nor manufactured before receiving surgeon approval of the surgical plan. Without being bound by theory, waiting to design the patient-specific implant until after the surgeon approves the surgical plan may increase the efficiency of the method 500 and/or reduce the resources necessary to perform the method 500. In other embodiments, one or more patient-specific implants can be designed and included in the surgical plans transmitted to the surgeon at block 506. Accordingly, in some embodiments the operation at block 514 can be included within the block 504.

The method 500 can continue at block 516 by manufacturing the patient-specific implant. The implant can be manufactured using additive manufacturing techniques, such as 3D printing, stereolithography, digital light processing, fused deposition modeling, selective laser sintering, selective laser melting, electronic beam melting, laminated object manufacturing, powder bed printing, thermoplastic printing, direct material deposition, or inkjet photo resin printing, or like technologies, or combination thereof. Alternatively or additionally, the implant can be manufactured using subtractive manufacturing techniques, such as CNC machining, electrical discharge machining (EDM), grinding, laser cutting, water jet machining, manual machining (e.g., milling, lathe/turning), or like technologies, or combinations thereof. The implant may be manufactured by any suitable manufacturing system (e.g., the manufacturing system 124 shown in FIG. 1 or the manufacturing system 950 described below with respect to FIG. 9). In some embodiments, the implant is manufactured by the manufacturing system executing the computer-readable fabrication instructions generated by the computing system at block 516.

Once the implant is manufactured at block 516, the method 500 can continue at block 518 by performing the selected surgical plan and implanting the patient-specific implant into the patient. Aspects of the surgical plan, such as some or all of the surgical procedure, can be performed manually, by a robotic surgical platform (e.g., a surgical robot), or a combination thereof. In embodiments in which the surgical procedure is performed at least in part by a robotic surgical platform, the surgical plan can include computer-readable control instructions configured to cause the surgical robot to perform, at least partly, the patient-specific surgical procedure.

In some embodiments, surgical outcomes can be collected and used to train the machine learning models used to perform various operations of the method 500. For example, the method 500 can optionally include receiving post-operative diagnostic scores that evaluate the predicted outcomes. Predicted test results for tests discussed in connection with FIGS. 4A and 4B can be generated using one or more machine learning algorithms. The patient can be tested periodically after surgery to collect post-operative diagnostic, which can be used to retrain the machine learning algorithms to increase machine learning accuracy. The post-operative diagnostic data can be compared to corresponding pre-operative diagnostic data to evaluate treatment efficacy, outcomes, etc. The post-operative diagnostic data can also be compared to corresponding predicted post-operative outcomes to evaluate accuracy of pre-operative predictions.

The method 500 can be implemented and performed in various ways. In some embodiments, the operations at blocks 502-514 can be performed by a computing system associated with a first entity, block 516 can be performed by a manufacturing system associated with a second entity, and block 518 can be performed by a surgical provider, surgeon, and/or robotic surgical platform associated with a third entity. Any of the foregoing blocks may also be implemented as computer-readable instructions stored in memory and executable by one or more processors of the associated computing system(s).

### D. Conclusion

As one skilled in the art will appreciate, any of the software modules described previously may be combined into a single software module for performing the operations described herein. Likewise, the software modules can be distributed across any combination of the computing systems and devices described herein, and are not limited to the express arrangements described herein. Accordingly, any of the operations described herein can be performed by any of the computing devices or systems described herein, unless expressly noted otherwise.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2018, titled “SYSTEMS AND METHODS FOR ASSISTING AND AUGMENTING SURGICAL PROCEDURES;”
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”
U.S. application Ser. No. 16/207,116, filed on Dec. 1, 2018, titled “SYSTEMS AND METHODS FOR MULTI-PLANAR ORTHOPEDIC ALIGNMENT;”
U.S. application Ser. No. 16/352,699, filed on Mar. 13, 2019, titled “SYSTEMS AND METHODS FOR ORTHOPEDIC IMPLANT FIXATION;”
U.S. application Ser. No. 16/383,215, filed on Apr. 12, 2019, titled “SYSTEMS AND METHODS FOR ORTHOPEDIC IMPLANT FIXATION;”
U.S. application Ser. No. 16/569,494, filed on Sep. 12, 2019, titled “SYSTEMS AND METHODS FOR ORTHOPEDIC IMPLANTS;”
U.S. application Ser. No. 16/699,447, filed Nov. 29, 2019, titled “SYSTEMS AND METHODS FOR ORTHOPEDIC IMPLANTS;”
U.S. application Ser. No. 16/735,222, filed Jan. 6, 2020, titled “PATIENT-SPECIFIC MEDICAL PROCEDURES AND DEVICES, AND ASSOCIATED SYSTEMS AND METHODS;”
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”
U.S. application Ser. No. 17/085,564, filed Oct. 30, 2020, titled “SYSTEMS AND METHODS FOR DESIGNING ORTHOPEDIC IMPLANTS BASED ON TISSUE CHARACTERISTICS;”
U.S. application Ser. No. 17/100,396, filed Nov. 20, 2020, titled “PATIENT-SPECIFIC VERTEBRAL IMPLANTS WITH POSITIONING FEATURES;”
U.S. application Ser. No. 17/342,439, filed Jun. 8, 2021, titled “PATIENT-SPECIFIC MEDICAL PROCEDURES AND DEVICES, AND ASSOCIATED SYSTEMS AND METHODS;”
U.S. application Ser. No. 17/463,054, filed Aug. 31, 2021, titled “BLOCKCHAIN MANAGED MEDICAL IMPLANTS;”
U.S. application Ser. No. 17/518,524, filed Nov. 3, 2021, titled “PATIENT-SPECIFIC ARTHROPLASTY DEVICES AND ASSOCIATED SYSTEMS AND METHODS;”
U.S. application Ser. No. 17/531,417, filed Nov. 19, 2021, titled “PATIENT-SPECIFIC JIG FOR PERSONALIZED SURGERY;”
U.S. application Ser. No. 17/678,874, filed Feb. 23, 2022, titled “NON-FUNGIBLE TOKEN SYSTEMS AND METHODS FOR STORING AND ACCESSING HEALTHCARE DATA;”
U.S. application Ser. No. 17/835,777, filed Jun. 8, 2022, titled “PATIENT-SPECIFIC EXPANDABLE INTERVERTEBRAL IMPLANTS;”
U.S. application Ser. No. 17/842,242, filed Jun. 16, 2022, titled “PATIENT-SPECIFIC ANTERIOR PLATE IMPLANTS;”
U.S. application Ser. No. 17/851,487, filed Jun. 28, 2022, titled “PATIENT-SPECIFIC ADJUSTMENT OF SPINAL IMPLANTS, AND ASSOCIATED SYSTEMS AND METHODS;”
U.S. application Ser. No. 17/856,625, filed Jul. 1, 2022, titled “SPINAL IMPLANTS FOR MESH NETWORKS;”
U.S. application Ser. No. 17/867,621, filed Jul. 18, 2022, titled “PATIENT-SPECIFIC SACROILIAC IMPLANT, AND ASSOCIATED SYSTEMS AND METHODS;”
U.S. application Ser. No. 17/868,729, filed Jul. 19, 2022, titled “SYSTEMS FOR PREDICTING INTRAOPERATIVE PATIENT MOBILITY AND IDENTIFYING MOBILITY-RELATED SURGICAL STEPS;”
U.S. application Ser. No. 17/951,085, filed Sep. 22, 2022, titled “SYSTEMS FOR MANUFACTURING AND PRE-OPERATIVE INSPECTING OF PATIENT-SPECIFIC IMPLANTS;”
U.S. application Ser. No. 17/978,673, filed Nov. 1, 2022, titled “SPINAL IMPLANTS AND SURGICAL PROCEDURES WITH REDUCED SUBSIDENCE, AND ASSOCIATED SYSTEMS AND METHODS;”
U.S. application Ser. No. 17/978,746, filed Nov. 1, 2022, titled “PATIENT-SPECIFIC SPINAL INSTRUMENTS FOR IMPLANTING IMPLANTS AND DECOMPRESSION PROCEDURES;”
U.S. application Ser. No. 18/102,444, filed Jan. 27, 2023, titled “TECHNIQUES TO MAP THREE-DIMENSIONAL HUMAN ANATOMY DATA TO TWO-DIMENSIONAL HUMAN ANATOMY DATA;”
U.S. application Ser. No. 18/113,573, filed Feb. 24, 2023, titled “PATIENT-SPECIFIC IMPLANT DESIGN AND MANUFACTURING SYSTEM WITH A DIGITAL FILING CABINET;”
U.S. application Ser. No. 18/120,979, filed Mar. 13, 2023, titled “MULTI-STAGE PATIENT-SPECIFIC SURGICAL PLANS AND SYSTEMS AND METHODS FOR CREATING AND IMPLEMENTING THE SAME;”
U.S. application Ser. No. 18/455,881, filed Aug. 25, 2023, titled “SYSTEMS AND METHODS FOR GENERATING MULTIPLE PATIENT-SPECIFIC SURGICAL PLANS AND MANUFACTURING PATIENT-SPECIFIC IMPLANTS;”
U.S. application Ser. No. 18/384,762, filed Oct. 28, 2023, titled “SYSTEMS AND METHODS FOR SELECTING, REVIEWING, MODIFYING, AND/OR APPROVING SURGICAL PLANS;”
U.S. application Ser. No. 18/537,600, filed Dec. 12, 2023, titled “PATIENT-SPECIFIC IMPLANT DESIGN AND MANUFACTURING SYSTEM WITH A REGULATORY AND REIMBURSEMENT MANAGER;”
International Application No. PCT/US24/10202, filed Jan. 3, 2024, titled “PATIENT-SPECIFIC SPINAL FUSION DEVICES AND ASSOCIATED SYSTEMS AND METHODS;”
U.S. application Ser. No. 18/408,409, filed Jan. 9, 2024, titled “SYSTEM FOR EDGE CASE PATHOLOGY IDENTIFICATION AND IMPLANT MANUFACTURING;”
U.S. application Ser. No. 18/408,452, filed Jan. 9, 2024, titled “SYSTEM FOR MODELING PATIENT SPINAL CHANGES;”
U.S. application Ser. No. 18/415,577, filed Jan. 17, 2024, titled “PATIENT-SPECIFIC IMPLANT DESIGN AND MANUFACTURING SYSTEM WITH A SURGICAL IMPLANT POSITIONING MANAGER;”
U.S. Application No. 63/530,427, filed Aug. 2, 2023, titled “MEDICAL DEVICE INSERTER INSTRUMENTS WITH RETRACTABLE COUPLING ELEMENTS AND METHODS OF USING THE SAME;”
U.S. Application No. 63/539,797, filed Sep. 21, 2023, titled “ROTATABLE INTERVERTEBRAL IMPLANTS FOR TRANSFORAMINAL LUMBAR INTERBODY FUSION TECHNIQUES;” and
U.S. Application No. 63/542,264, filed Oct. 3, 2023, titled “PATIENT-SPECIFIC SURGICAL POSITIONING GUIDES AND METHODS OF MAKING AND USING THE SAME.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

The ranges disclosed herein also encompass any and all overlap, sub-ranges, and combinations thereof. Language such as “up to,” “at least,” “greater than,” “less than,” “between,” or the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed is:

1. A method comprising:
obtaining diagnostic data for a patient, the diagnostic data including image data;
generating a virtual model representing the patient's spine based on the image data, wherein the virtual model has movable anatomic spinal elements;
generating a biomechanical virtual model of the patient's spine based on the virtual model; and
determining one or more spinal metrics based on the biomechanical virtual model for viewing by a user.

2. The method of claim 1, further comprising:
determining one or more motion parameters associated with the spine for simulating at least one of flexion, extension, rotation, and lateral flexion, wherein the one or more motion parameters are derived from the diagnostic data; and
assigning the determined one or more motion parameters to the virtual model to generate at least a portion of the biomechanical virtual model.

3. The method of claim 2 wherein the one or more motion parameters are determined from at least one of a gait test, a motion test, or a spinal stability test.

4. The method of claim 2 wherein:
the one or more motion parameters are associated with two or more anatomic spinal elements of the patient, and
the determined one or more motion parameters are assigned to anatomical spinal elements of the biomechanical virtual model corresponding to the two or more anatomic spinal elements of the patient.

5. The method of claim 1, further comprising designing an implant based on the biomechanical virtual model.

6. The method of claim 1, further comprising
generating a surgical plan based on the biomechanical virtual model; and
transmitting the surgical plan for viewing by a user, wherein the surgical plan includes information from the biomechanical virtual model.

7. The method of claim 1, further comprising:
determining one or more candidate modifications for the virtual model based on the images of the image data;
determining diagnostic data needed for the one or more candidate modifications; and
sending a request for the determined diagnostic data.

8. The method of claim 7, further comprising:
determining one or more modifications to the virtual model based on an orientation of the patient's spine when the image data was captured, wherein the one or more modifications compensate for state of patient's spine when the image data was captured.

9. The method of claim 1, further comprising:
validating motion of the biomechanical virtual model based on reference diagnostic data and matching diagnostic data of the patient.

10. The method of claim 1 wherein the one or more spinal metrics include at least one of a lumbar lordosis angle, Cobb angle, pelvic incidence measurement, disc height, or segment flexibility measurement.

11. The method of claim 1, further comprising
generating set of anatomical positions for the biomechanical virtual model;
positioning the biomechanical virtual model in each of the anatomical positions; and
measuring the biomechanical virtual model at each of the anatomical positions.

12. The method of claim 11 wherein the anatomical positions include a supine position, prone position, standing position, under flexion position, under extension position, neutral position, or side-bending position.

13. The method of claim 1 wherein the biomechanical model is a diagnostic-driven virtual model including
virtual anatomical elements, and
one or more diagnostic data-derived relationships associated with the virtual anatomical element.

14. A method for treating a patient, comprising
obtaining diagnostic data related to a patient's spine;
creating a virtual spine model based on the obtained diagnostic data;
simulating one or more surgical procedures on the virtual spine model to generate simulated procedure data;
comparing the simulated procedure data with reference diagnostic data to determine a surgical procedure for the patient; and
generating a surgical plan based on the determine surgical procedure.

15. The method of claim 14 wherein the reference diagnostic data is prior patient data of patients that are similar to the patient.

16. A computer-implemented method for treating a patient, the method comprising:
receiving patient demographic information and patient health information;
automatically selecting one or more diagnostic tests to be performed on the patient based at least in part on the patient demographic information and patient health information, the diagnostic tests selected to identify specific diagnostic information for use in determining a patient-specific treatment plan for the patient; and
generating a patient specific diagnostic plan, the patient-specific diagnostic plan identifying the one or more diagnostic tests and including instructions for performing the one or more diagnostic tests.

17. The computer-implemented method of claim 16 wherein selecting the one or more diagnostic tests includes:
comparing the patient demographic information and the patient health information to reference patients to identify a subset of the reference patients having similar demographic information and health information to the patient; and
selecting the one or more tests based at least in part on one or more tests identified as useful for determining a patient-specific treatment plan for the subset of the reference patients.

18. The computer-implemented method of claim 16 wherein selecting the one or more diagnostic tests includes using a trained machine learning model to analyze the patient demographic information and the patient health information to identify the one or more diagnostic tests.

19. The computer-implemented method of claim 16 wherein—
the patient demographic information includes one or more of patient age, sex, height, weight, or body mass index; and
the patient health information includes one or more of patient condition, patient symptoms, or patient medical history.

20. The computer-implemented method of claim 16, further comprising:
receiving diagnostic equipment information for a healthcare provider associated with treating the patient,
wherein automatically selecting the one or more diagnostic tests is further based in part on the diagnostic equipment information.

21. The computer-implemented method of claim 16 wherein the one or more diagnostic tests are selected based at least in part to obtain patient image data for generating a three-dimensional virtual model of patient anatomy.

22. The computer-implemented method of claim 16, further comprising determining a timeframe for performing the one or more diagnostic tests, wherein the patient-specific diagnostic plan further includes the timeframe.

23. The computer-implemented method of claim 16, further comprising receiving diagnostic information collected via execution of the patient-specific diagnostic plan.

24. The computer-implemented method of claim 23, further comprising automatically analyzing the received diagnostic information to extrapolate additional diagnostic information from the received diagnostic information.

25. The computer-implemented method of claim 24 wherein the received diagnostic information includes image data, and wherein the additional diagnostic information includes one or more physiological metrics extrapolated from the image data.

26. The computer-implemented method of claim 23, further comprising generating a virtual model of patient anatomy based at least in part on the diagnostic information.

27. The computer-implemented method of claim 23, further comprising:
analyzing the received diagnostic information, and
in response to a determination that the received diagnostic information is insufficient to determine a treatment plan, identifying one or more additional diagnostic tests to be performed on the patient, and
transmitting the one or more additional diagnostic tests for execution.

28. The computer-implemented method of claim 27 wherein the operations of receiving the diagnostic information, analyzing the received diagnostic information, identifying the one or more additional diagnostic tests, and transmitting the one or more additional diagnostic tests are performed in substantially real time.

29. The computer-implemented method of claim 23, further comprising determining the patient-specific treatment plan based at least in part on the received diagnostic information.

30. The computer-implemented method of claim 29 wherein determining the patient-specific treatment plan includes generating a virtual biomechanical model of patient anatomy based on the diagnostic information.

31. A system for providing patient-specific medical care, the system comprising:
one or more processors; and
a memory storing instructions that, when executed by the one or more processors, cause the system to perform operations comprising:
receiving patient demographic information and patient health information,
automatically selecting one or more diagnostic tests to be performed on the patient based at least in part on the patient demographic information and patient health information, the diagnostic tests selected to identify specific diagnostic information for use in determining a patient-specific treatment plan for the patient, and
generating a patient specific diagnostic plan, the patient-specific diagnostic plan identifying the one or more diagnostic tests and including instructions for performing the one or more diagnostic tests.

32. The system of claim 31 wherein the operation of automatically selecting the one or more diagnostic tests includes:
comparing the patient demographic information and the patient health information to reference patients to identify a subset of the reference patients having similar demographic information and health information to the patient; and
selecting the one or more tests based at least in part on one or more tests identified as useful for determining a patient-specific treatment plan for the subset of the reference patients.

33. The system of claim 32 wherein the operation of selecting the one or more diagnostic tests includes using a trained machine learning model to analyze the patient demographic information and the patient health information to identify the one or more diagnostic tests.

34. The system of claim 31 wherein—
the patient demographic information includes one or more of patient age, sex, height, weight, or body mass index; and
the patient health information includes one or more of patient condition, patient symptoms, or patient medical history.

35. The system of claim 31 wherein the operations further comprise:
receiving diagnostic equipment information for a healthcare provider associated with treating the patient,
wherein automatically selecting the one or more diagnostic tests is further based in part on the diagnostic equipment information.

36. The system of claim 31 wherein the one or more diagnostic tests are selected based at least in part to obtain patient image data for generating a three-dimensional virtual model of patient anatomy.

37. The system of claim 31 wherein the operations further comprise determining a timeframe for performing the one or more diagnostic tests, wherein the patient-specific diagnostic plan further includes the timeframe.

38. The system of claim 31 wherein the operations further comprise receiving diagnostic information collected via execution of the patient-specific diagnostic plan.

39. The system of claim 38 wherein the operations further comprise automatically analyzing the received diagnostic information to extrapolate additional diagnostic information from the received diagnostic information.

40. The system of claim 39 wherein the received diagnostic information includes image data, and wherein the additional diagnostic information includes one or more physiological metrics extrapolated from the image data.

41. The system of claim 38 wherein the operations further comprise generating a virtual model of patient anatomy based at least in part on the diagnostic information.

42. The system of claim 38 wherein the operations further comprise:
analyzing the received diagnostic information, and
in response to a determination that the received diagnostic information is insufficient to determine a treatment plan, identifying one or more additional diagnostic tests to be performed on the patient, and
transmitting the one or more additional diagnostic tests for execution.

43. The system of claim 42 wherein the operations of receiving the diagnostic information, analyzing the received diagnostic information, identifying the one or more additional diagnostic tests, and transmitting the one or more additional diagnostic tests are performed in substantially real time.

44. The system of claim 38 wherein the operations further comprise determining the patient-specific treatment plan based at least in part on the received diagnostic information.

45. The system of claim 44 wherein the operation of determining the patient-specific treatment plan includes generating a virtual biomechanical model of patient anatomy based on the diagnostic information.
